(19) United States
(12) Patent Application Publication
(10) Pub. No.: US 2016/0022869 A1
(43) Pub. Date: Jan. 28, 2016

(54) BIOLOGICAL FUNCTIONALISATION OF SUBSTRATES

(71) Applicant: THE UNIVERSITY OF SYDNEY, Sydney (AU)

(72) Inventors: Marcela Bilek, Sutherland (AU); David McKenzie, Artarmon (AU); Yongbai Yin, Epping (AU)

(73) Assignee: THE UNIVERSITY OF SYDNEY, Sydney (AU)

(21) Appl. No.: 14/872,188

(22) Filed: Oct. 1, 2015

Related U.S. Application Data
(63) Continuation of application No. 12/452,882, filed on Apr. 5, 2010, now abandoned.

(30) Foreign Application Priority Data
Jul. 27, 2007 (AU) 2007904052

(51) Int. Cl. A61L 27/34; G01N 33/543; A61L 27/04

(52) U.S. Cl. CPC A61L 27/34; A61L 27/042; G01N 33/54306; A61L 2300/252; A61L 2400/18; G01N 2333/908

(57) ABSTRACT

The invention relates to an activated metallic, semiconductor, polymer, composite and/or ceramic substrate, the substrate being bound through a mixed or graded interface to a hydrophilic polymer surface that is activated to enable direct covalent binding to a functional biological molecule, the polymer surface comprising a sub-surface that includes a plurality of cross-linked regions, as well as to such activated substrates that have been functionalised with a biological molecule and to devices comprising such functionalised substrates. Such substrates can be produced by a method comprising steps of: a. exposing a surface of the substrate to any or more of (i) to (iii): (i) plasma ion implantation with carbon containing species; (ii) co-deposition under conditions in which substrate material is deposited with carbon containing species while gradually reducing substrate material proportion and increasing carbon containing species proportion; (iii) deposition of a plasma polymer surface layer with energetic ion bombardment; incubating the surface treated according to step (a) with a desired biological molecule.

### FIELD OF THE INVENTION

The present invention relates in particular, but not exclusively, to activated substrates capable of binding functional biological molecules, to substrates comprising bound and functional biological molecules, to devices comprising such substrates and to methods of producing them. In particular, the activated substrates comprise metals, semiconductors, polymers, composite materials and/or ceramics.

### BACKGROUND OF THE INVENTION

The advent of diagnostic array technology (where for example protein, antibody or other biological molecule/s is/are attached at discrete locations on a substrate surface to allow attachment of other molecules of interest (target molecules) and where means for detecting the attachment of the target molecules is provided) has led to an increased demand for surfaces capable of binding to biological molecules such as antibodies, other proteins and nucleic acids. It is similarly necessary in other applications, such as for example biosensors, medical devices where biocompatible surfaces are required and in the screening of active agents against drug targets, that surfaces capable of binding to biological molecules are required.

An ideal surface for these applications should bind proteins or other biological molecules while preserving their functionality. The binding is preferably strong and stable over extended periods to allow repeated washing steps during processing. In many of these technologies the protein (or other biological molecule) binding to the substrate surface is attached through non-specific physisorption, leading to losses of protein during washing and variability in the degree of attachment given that the attachment process is molecular species dependent. Functionality of physisorbed proteins depends strongly on the energetics of the interaction with the surface and will vary across proteins.

It is of interest to be able to attach biological molecules strongly, preferably by means of a covalent bond, to surfaces of metals, semiconductors, polymers, composite materials and/or ceramics in a variety of applications. For example, metals have desirable strength and elastic properties that make them suitable for use in repairing human and animal bones and joints. In particular, metal prosthetic pins and plates can be used to repair bone after fracture. In this context it is desirable to attach bone cells firmly to the metal surface so that the metal part is firmly anchored in the skeleton. For such applications it is desirable to promote the healthy growth of osteoblasts and to suppress growth of fibroblasts that give rise to fibrous tissue. Such differentiation of cell attachment can be facilitated by attaching to the surface one or more suitable biologically active molecules. Another application of a metal prosthetic part is in stents for maintaining flow through blood vessels or other body cavities. Such devices should be biocompatible but should not promote excessive fibrous tissue or smooth muscle cell growth, whilst promoting the attachment and growth of endothelial cells. Such differentiation can also be attained by attaching suitable biological molecules to the metal surface.

It is also desirable to be able to covalently attach biological molecules to the surfaces of ceramics for purposes of skeletal repair, for the same reasons as outlined above in relation to metals. Indeed there are a variety of other contexts in which it is desirable to be able to covalently attach biological molecules to the surfaces of metals, ceramics, semiconductors, polymers or to the surfaces of composite materials that have some metallic, ceramic, polymeric and/or semiconductor characteristics or features. For example, it is desirable to attach biological molecules to surfaces in the contexts of assays and detection devices, scaffolds for tissue and/or organ generation, screening of compounds for useful biological activity, micro- and nano-devices that interact with or include biological components (e.g. molecular motors involving actin/myosin filaments), fuel cells that incorporate a biological processing component (e.g. fuel cells comprising photosynthetic cells). A further specific example is that of semiconductors that can be used for the detection of biological molecules by sensing the specific attachment of the target molecules to detection molecules bound on the semiconductor surface. In this context the attachment of such a detection molecule to the semiconductor surface with a permanent and strong bond, preferably a covalent bond. The function of the detection molecule in recognising its target molecule should not be compromised by the attachment process.

A number of groups have conducted work in relation to use of plasma gas treatments to attach biological molecules to polymer surfaces. However, these methods have not been useful for attaching functional biological molecules to metal, semiconductor, ceramic or composite substrates. Known methods for attaching protein to such substrates involve the use of linker molecules such as thiol linker molecules on gold surfaces (bonding via a sulfur-gold interaction) or the use of simple carbon chain linker molecules that are bound to specific functional groups on the surface. These methods involve first covering the surface with gold or the functional group required (often amines or carboxyl groups, for example), effecting the attachment of linker molecules using solution chemistry, and then attaching the proteins to the linkers. The present inventors are not aware of any current options available for attaching functional biological molecules to metal, semiconductor or ceramic surfaces that do not require the addition of a linker molecule.

The functionalising of surfaces by deposition of a plasma polymer has been reported. However, the disadvantage of simple deposition of a plasma polymer is that the adhesion is generally poor and the surface will delaminate, especially in solution or where the surface is exposed to some stress.

The present inventors have devised a method that can be used to covalently bind functional biological molecules to a substrate, especially metal, semiconductor, polymer, ceramic or composite substrates, without the need to use linker molecules (and therefore without associated wet chemistry). In one embodiment the invention involves a simple two step plasma modification process including ion implantation and/or deposition, to create a mixed or graded interface, followed by the deposition of a hydrophilic plasma polymer. The binding of biological molecules then involves simple adsorption (resulting in covalent binding), with no further chemistry required.

It is with the above background in mind that the present invention has been conceived.

### SUMMARY OF THE INVENTION

According to one embodiment of the present invention there is provided an activated metallic, semiconductor, polymer, composite and/or ceramic substrate, the substrate being bound through a mixed or graded interface to a hydrophilic plasma polymer surface that is activated to enable direct covalent binding to a functional biological molecule, the plasma polymer surface comprising a sub-surface that includes a plurality of cross-linked regions.

According to another embodiment of the present invention there is provided a functionalised metallic, semiconductor, polymer, composite and/or ceramic substrate, the substrate being bound through a mixed or graded interface to a hydrophilic plasma polymer surface that is directly covalently bound to a functional biological molecule, the plasma polymer surface comprising a sub-surface that includes a plurality of cross-linked regions.

According to another embodiment of the present invention there is provided a device comprising an activated metallic, semiconductor, polymer, composite and/or ceramic substrate, the substrate being bound through a mixed or graded interface to a hydrophilic plasma polymer surface that is activated to enable direct covalent binding to a functional biological molecule, the plasma polymer surface comprising a sub-surface that includes a plurality of cross-linked regions.

According to another embodiment of the present invention there is provided a device comprising a functionalised metallic, semiconductor, polymer, composite and/or ceramic substrate, the substrate being bound through a mixed or graded interface to a hydrophilic plasma polymer surface that is directly covalently bound to a functional biological molecule, the plasma polymer surface comprising a sub-surface that includes a plurality of cross-linked regions.

According to another embodiment of the present invention there is provided a method of producing an activated metallic, semiconductor, polymer, composite and/or ceramic substrate, the substrate being bound through a mixed or graded interface to a hydrophilic plasma polymer surface that is activated to enable direct covalent binding to a functional biological molecule, the plasma polymer surface comprising a sub-surface that includes a plurality of cross-linked regions, comprising exposing a surface of the substrate to any or more of (i) to (iii):
(i) plasma ion implantation with carbon containing species;
(ii) co-deposition under conditions in which substrate material is deposited with carbon containing species while gradually reducing substrate material proportion and increasing carbon containing species proportion;
(iii) deposition of a plasma polymer surface layer with energetic ion bombardment.

According to a further embodiment of the present invention there is provided a method of producing a functionalised metallic, semiconductor, polymer, composite and/or ceramic substrate, the substrate being bound through a mixed or graded interface to a hydrophilic plasma polymer surface that is directly covalently bound to a functional biological molecule, the plasma polymer surface comprising a sub-surface that includes a plurality of cross-linked regions, comprising steps of:
(a) exposing a surface of the substrate to any or more of (i) to (iii):
   (i) plasma ion implantation with carbon containing species;
   (ii) co-deposition under conditions in which substrate material is deposited with carbon containing species while gradually reducing substrate material proportion and increasing carbon containing species proportion;
   (iii) deposition of a plasma polymer surface layer with energetic ion bombardment;
(b) incubating the surface treated according to step (a) with a desired biological molecule.

According to another embodiment of the present invention there is provided an activated metallic, semiconductor, polymer, composite and/or ceramic substrate, the substrate being bound through a mixed or graded interface to a hydrophilic plasma polymer surface that is activated to enable direct covalent binding to a functional biological molecule, the plasma polymer surface comprising a sub-surface that includes a plurality of cross-linked regions, the activated substrate produced according to a method comprising exposing a surface of the substrate to any or more of (i) to (iii):
(i) plasma ion implantation with carbon containing species;
(ii) co-deposition under conditions in which substrate material is deposited with carbon containing species while gradually reducing substrate material proportion and increasing carbon containing species proportion;
(iii) deposition of a plasma polymer surface layer with energetic ion bombardment.

According to a still further embodiment of the present invention there is provided a functionalised metallic, semiconductor, polymer, composite and/or ceramic substrate, the substrate being bound through a mixed or graded interface to a hydrophilic plasma polymer surface that is directly covalently bound to a functional biological molecule, the plasma polymer surface comprising a sub-surface that includes a plurality of cross-linked regions, the functionalised substrate produced according to a method comprising steps of:
(a) exposing a surface of the substrate to any or more of (i) to (iii):
   (i) plasma ion implantation with carbon containing species;
   (ii) co-deposition under conditions in which substrate material is deposited with carbon containing species while gradually reducing substrate material proportion and increasing carbon containing species proportion;
   (iii) deposition of a plasma polymer surface layer with energetic ion bombardment;
(b) incubating the surface treated according to step (a) with a desired biological molecule.

### BRIEF DESCRIPTION OF THE FIGURES

The invention will be further described with reference to the figures, wherein:

FIG. 1 shows a schematic diagram of the capacitively coupled plasma treatment chamber with facility for controlling substrate bias, useful in methods of activating surfaces of metal, semiconductor, polymer, composite and/or ceramic substrates according to the invention.

FIG. 2 shows a schematic diagram of the capacitively coupled plasma treatment chamber with magnetron source for co-deposition, useful in methods of activating surfaces of metal, semiconductor, polymer, composite and/or ceramic substrates according to the invention.

FIG. 3 shows a schematic diagram of the inductively coupled plasma treatment chamber useful in methods of activating surfaces of metal, semiconductor, polymer, composite and/or ceramic substrates according to the invention.

FIG. 4 compares FTIR data from a HRP coated sample (sample P11) before and after washing with SDS detergent which disrupts all interactions except for covalent bonding. Absorbance as measured by integrated counts from the Amide A, Amide 1 and Amide 2 spectral regions which were normalised by integrated counts from the CH stretching vibration at 2917 cm⁻¹ and plotted as the columns. The left hand blue coloured columns show the values obtained from spectra of the HRP coated plasma polymerised surfaces and the right hand violet coloured columns show the values for the same surfaces after SDS washing.

FIG. 5 shows the degree of activity retained by the protein as a function of time after incubation in protein containing solution for plasma polymerised samples (samples P11 and P20). Results of the TMB assay from plasma immersion ion implanted polyethylene sheet and untreated polyethylene sheet incubated in HRP solution were used as controls and are shown for comparison. The activity was measured by a HRP activity assay using TMB reactant.

FIG. 6 shows FTIR spectra of hexane plasma polymerized films on gold coated glass, deposited with and without bias on the sample electrode. Spectra correspond to the samples PE5 and PE6, respectively (Table 1).

FIG. 7 shows FTIR ATR difference spectra showing HRP attached to plasma polymerized hexane coatings on polyethylene substrates. Spectra taken from the coating before protein attachment are subtracted from that taken after incubation in protein containing solution and subsequent washing.

FIG. 8 shows normalised absorbance due to attached HRP protein molecules as a function of the carbonyl peak absorbance as measured by FTIR ATR. Squares—immediately after incubation in protein solution and washing in fresh buffer; triangles—after SDS washing; solid symbols—on untreated polyethylene; gray symbols—coating prepared on the grounded electrode. Solid lines serve as a guide to the eye only.

FIG. 9 shows HRP bioactivity as determined by TMB colorimetric assays for HRP attached on plasma polymerized hexane coatings (rhombi—typical coatings deposited on negative self biased electrodes; squares—untreated polyethylene; circles—plasma polymerized hexane coating prepared with the substrate on the grounded electrode).

FIG. 10 shows FTIR ATR difference spectra of plasma polymerized hexane coatings on polyethylene substrates. Spectra were taken (1) before the incubation of the surfaces in HRP containing buffer and rinsing and then again (2) after incubation and subsequent washing in SDS detergent solution. Spectra of coating before protein attachment are subtracted from that after the SDS rinse (ie (2)-(1) is shown).

FIG. 11 shows a schematic diagram of the plasma polymerization system used for deposition from acetylene.

FIG. 12 shows a bar graph demonstrating HRP enzyme surface activity after immobilization on silicon substrate, polyethylene surface (as controls), and an acetylene plasma deposited surface at day 0, 5, and 11 respectively.

FIG. 13 shows the RC time constant as a function of the incubation time of soybean peroxidase by injecting the enzyme into the PB solution cell, in which the active electrode was an acetylene plasma deposited surface on a doped silicon substrate.

FIG. 14 shows the HRP activities for day 0 and day 5 respectively after HRP attachment on acetylene plasma deposited surfaces, showing an as-prepared plasma deposited surface (Denoted as “Control”), the plasma deposited surface after oxygen plasma etching (denoted as “Oxygen Plasma”), the plasma deposited surface after argon plasma etching (denoted as “Argon Plasma”), the plasma deposited surface after 380° C. anneal for 30 minutes (denoted as 380 C Anneal”), and the plasma deposited surface after oxygen plasma etching followed by 420° C. anneal for 30 minutes (denoted as “Oxygen Plasma+420 C Anneal”).

FIG. 15 shows the HRP activity on two acetylene plasma deposited surfaces produced in the same batch and stored for different shelf times in ambient environment prior to incubation in protein solution. Data from a colorimetric activity assay performed 0 and 6 days after immobilization are shown.

FIG. 16 shows absorbance at 450 nm from a TMB assay for horseradish peroxidase activity: “N2 Plasma” refers to acetylene plasma deposited coatings made with 8 SCCM nitrogen gas flow; “Ar Plasma” refers to acetylene plasma deposited coatings made with 8 SCCM argon flow; “N2+Ar Plasma” refers to acetylene plasma deposited coatings made with 4 SCCM flow of both nitrogen and argon; and “SS” refers to the uncoated 316L stainless steel surface.

FIG. 17 shows mass change during QCM-D analysis on an acetylene plasma deposited surface during the surface immobilization of tropoelastin. The point labeled “Tropo” indicates the switch from Phosphate Buffer to Tropoelastin solution. The label “PB” denotes a switch back to fresh phosphate buffer. The on-set of SDS detergent flow is indicated by “SDS” and the change due to ethanol flow is indicated by “Ethanol”.

FIG. 18 shows the dissipation change in QCM-D analysis on acetylene plasma deposited surface during tropoelastin immobilization, phosphate buffer (PB) washing, SDS cleaning, and ethanol cleaning.

FIG. 19 shows the dissipation change in QCM-D analysis as a function of attached mass on an acetylene plasma deposited surface during tropoelastin immobilization, phosphate buffer (PB) washing, SDS cleaning, and ethanol cleaning.

FIG. 20 shows raw ellipsometry data before (thin lines) and after (thick lines) tropoelastin attachment onto an acetylene plasma deposited surface followed by thorough rinsing in fresh phosphate buffered saline (PBS). The 75° and 65° data are offset by −5 and 5 degrees respectively for improved readability.

FIG. 21 shows detection of attached tropoelastin before and after SDS cleaning using an antibody assay (ELISA). “SS” indicates results for an argon plasma cleaned stainless steel surface, while “PD surface” indicates those for the surface coating deposited from acetylene plasma and “no tropo” refers to a control surface with no tropoelastin.

FIG. 22 shows the absorbance measured in an HRP activity assay for day 0 and day 7 after incubation in HRP on (from left) an acetylene plasma deposited surface used as a control; the same surface after annealing at 350 C in vacuum; and a PIII treated polyethylene control.

FIG. 23 shows a comparison of tropoelastin attachment on acetylene plasma deposited with a range of applied negative substrate bias from 0 to −1 kV as measured by an ELISA assay before and after SDS cleaning. PIII and untreated polystyrene surfaces as well as argon plasma cleaned stainless steel are included as controls. The surfaces were incubated in tropoelastin after deposition and SDS detergent washing was used to test the strength of binding of the tropoelastin. This shows that the binding of proteins to the films is much higher than for untreated polystyrene surfaces and stainless steel.

FIG. 24 shows the change of polar and dispersic surface energies as a function of storage time in laboratory atmosphere.

FIG. 25 shows the dependence of the electron spin density in acetylene plasma deposited surfaces as measured by electron spin resonance (ESR) on the pulsed bias voltage used during deposition. The Day 4 results refer to samples aged in laboratory air for four days and Day 25 results refer to samples aged in laboratory air for 25 days. Day 25 samples show reduced spin density. After annealing of the Day 25 samples in vacuum at 320 C for 20 minutes, the spin density returns to values close to those for the Day 4 samples.

FIG. 26 shows the activity of HRP on acetylene plasma deposited surfaces co-deposited with magnetron sputtered stainless steel prepared in the system of FIG. 2. The flow rate of acetylene in the acetylene/argon process gas mixture was varied while the total pressure was kept constant. The horizontal axis labels refer to the acetylene flow rate in sccm. The last data, labelled “PIII”, refer to measurements on a PIII polyethylene control.

FIG. 27 shows a comparison of tropoelastin attachment before and after SDS cleaning on acetylene plasma deposited surfaces co-deposited with magnetron sputtered stainless steel prepared in the system of FIG. 2. The flow rate of acetylene in the acetylene/argon process gas mixture was varied while the total pressure was kept constant. The horizontal axis labels refer to the acetylene flow rate in sccm. The last data, labelled “UT PS” and “PIII PS”, refer to measurements on a untreated polystyrene control and a PIII treated polystyrene control.

### DETAILED DESCRIPTION OF THE INVENTION

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Documents referred to within this specification are included herein in their entirety by way of reference.

The reference to any prior art in this specification is not, and should not be taken as, an acknowledgment or any form of suggestion that that prior art forms part of the common general knowledge in Australia.

As mentioned above, in one broad embodiment this invention relates to an activated metallic, semiconductor, polymer, composite and/or ceramic substrate, the substrate being bound through a mixed or graded interface to a hydrophilic plasma polymer surface that is activated to enable direct covalent binding to a functional biological molecule, the plasma polymer surface comprising a sub-surface that includes a plurality of cross-linked regions. The invention also encompasses devices comprising such activated polymer substrates.

By the term “activated” it is intended to mean that the hydrophilic surface layer (which also results from the processes of the invention) on the treated metal, semiconductor, polymer, composite and/or ceramic substrate has been processed or generated in a manner such that it is able to accept a biological molecule for binding, upon exposure thereto. That is, the surface layer on the metal, semiconductor, polymer, composite and/or ceramic has one or more higher energy state regions where there are chemical groups or electrons available for participation in binding to one or more groups on a biological molecule, or indeed to suitable linker groups, which in turn are bound or are able to bind to a biological molecule.

In another broad aspect of the invention there is provided a functionalised metallic, semiconductor, polymer, composite and/or ceramic substrate, the substrate being bound through a mixed or graded interface to a hydrophilic plasma polymer surface that is directly covalently bound to a functional biological molecule, the plasma polymer surface comprising a sub-surface that includes a plurality of cross-linked regions. The invention also encompasses devices comprising such functionalised metal, semiconductor, polymer, composite and/or ceramic substrates.

Without wishing to be bound by theory, the present inventors believe that through the activation of the plasma polymer surface layer on the metal, semiconductor, polymer, composite and/or ceramic according to the invention it is possible to form chemical bonds, most likely covalent bonds, to chemical groups of biological molecules or linkers that attach to biological molecules. Preferably the chemical groups of the biological molecules are accessible for binding interactions, such as by being located on the exterior of the molecule. The present inventors believe that activation of the plasma polymer surface involves the generation of reactive free radicals or oxygen species, such as charged oxygen atoms and reactive carbonyl and carboxylic acid moieties that appear following exposure of the plasma treated or generated polymer surface to oxygen (e.g. from air), and which are then available as binding sites for reactive species on biological molecules, such as amine groups. The most likely mechanism to explain activation of the plasma polymer surface layer on the substrate is that the methods of the invention give rise to the generation of free radicals within the plasma polymer surface. Indicative of this mechanism is that while the biological activity of biological molecules with which the surface has been functionalised is retained over time, there appears to be a loss over time of the ability of activated surfaces to bind covalently to biological molecules. However, the ability to bind biological molecules to the activated surfaces can be regenerated (that is, the previously activated surfaces can effectively be re-activated) by adopting an annealing step. This is a step of applying energy to the surface, without destroying it, to allow molecular mobility within the plasma polymer such that buried free radicals can migrate to the surface where they can participate in covalent binding to biological molecules. Alternatively, the energy applied may release bound chemical species that, once released, give rise to free radicals. For example, annealing may be carried out by heating in an oven or exposure to steam or microwave energy (for example to temperatures of 250° C. to 400° C., 300° C. to 375° C., or approximately 350° C., depending upon the surface concerned). A preferred method of annealing is heating in a vacuum oven. The annealing step may be undertaken as part of the manufacture of the activated surface. For example this step may ensure that the activation is at a high level even if the manufacturing process is not fully optimised.

Within this application we refer to attachment of a biological molecule, or a linker for attachment to a biological molecule, as functionalisation of the plasma polymer surface on the metal, semiconductor, polymer, composite and/or ceramic substrate and to the plasma polymer surface on the metal, semiconductor, polymer, composite and/or ceramic substrate to which the biological molecule or linker is attached as being “functionalised”. Attachment by covalent bonds to an otherwise hydrophilic surface allows strong time stable attachment of biological molecules that are able to maintain a useful biological function. For example, the hydrophilic surface of the plasma polymer layer will ensure that it is not energetically favourable for proteins to denature on the surface. Covalent attachment to a surface can be achieved via amino acid side chain groups covalently attached to the surface or to linker molecules, for example. The strategy adopted is to prepare the plasma polymer surface with sites that encourage covalent attachment. In one approach a deposition process with energetic ion bombardment is utilised with the aim of stabilising the plasma polymer surfaces simultaneously with the creation of the binding sites. Using functionality assays, the inventors have demonstrated that associated with the adopted plasma surface treatment there is enhancement of functional protein attachment with covalent binding, compared to non-treated surfaces, as well as significantly increased resistance to repeated washing steps. That is, there is increased biological molecule binding relative to non-treated surfaces, the binding is strong and can withstand repeated washing and the molecule is able to retain useful activity (ie. the biological molecule is functional or retains some useful functionality).

By the term “functional” it is intended to convey that the molecule is able to exhibit at least some of the activity it would normally exhibit in a biological system. For example, activity may include the maintained ability to participate in binding interactions, such as antigen/antibody binding, receptor/drug binding, the maintained ability to catalyse or participate in a biological reaction or the ability to interact with cell membrane proteins in biological tissues even if this is at a lower level than is usual in a biological system. Routine assays are available to assess functionality of the biological molecule. Preferably the activity of the biological molecule bound to the activated plasma polymer surface is at least 20%, preferably at least 40%, more preferably at least 60%, 70% or 80% and most preferably at least 90%, 95%, 98% or 99% of the activity of the molecule when not bound to the activated plasma polymer surface. Most preferably the activity of the bound biological molecule is equivalent to that of a non-bound molecule.

By the term “biological molecule” it is intended to encompass any molecule that is derived from a biological source, is a synthetically produced replicate of a molecule that exists in a biological system, is a molecule that mimics the activity of a molecule that exists in a biological system or otherwise exhibits biological activity, or active fragments thereof. The term “biological molecule” also encompasses a combination or mixture of biological molecules. In the case of proteins (and a similar analogy can be made in the case of nucleic acids, carbohydrates or the like) active fragments are peptide sequences derived from the active protein that exhibit preferably at least at least 20%, preferably at least 40%, more preferably at least 60%, 70% or 80% and most preferably at least 90%, 95%, 98% or 99% of the activity of the active protein. Active peptide fragments are preferably at least 10, more preferably at least 15, more preferably at least 20, 30, 40 or 50 amino acids in length. Examples of biological molecules include, but are not limited to, amino acids, peptides, enzymes, proteins, glycoproteins, lipoproteins, nucleotides, oligonucleotides, nucleic acids (including DNA and RNA), lipids and carbohydrates, as well as active fragments thereof. Preferred biological molecules include proteins and drugs or drug targets. Particularly preferred biological molecules include antibodies and immunoglobulins, receptors, enzymes, neurotransmitters or other cell signalling agents, cytokines, hormones and complimentarity determining proteins, and active fragments thereof. The term “biological molecule” also encompasses molecules that are integral to or attached to cells or cellular components (eg. cell membrane proteins) through which cells or cellular components may be bound to the activated plasma polymer. Further specific examples of biological molecules included within the invention are toxins and poisons including naturally occurring toxins such as bacterial, viral, plant or animal derived toxins or active fragments thereof including conotoxin and snake and spider venoms, for example, and other organic or inorganic toxins and poisons such as cyanide and anti-bacterial, anti-fungal, herbicide and pesticide agents. A biological molecule of particular interest is tropoelastin, which is an extracellular matrix protein that can be used to functionalise surfaces to improve the biological compatibility of implantable or other devices. Enzymes of interest include those capable of breaking down cellulose into simple sugars such as cellulase.

An advantage associated with the present invention is that the process for binding biological molecules to the surface of a metal, semiconductor, polymer, composite and/or ceramic does not depend upon the specific biological molecule or metal, semiconductor, polymer, composite and/or ceramic and can therefore be applied to a wide variety of biological molecules and metals, semiconductors, polymers, composites and/or ceramics. Furthermore, and although it is possible for the biological molecules to be bound via a linker molecule, it is not necessary according to the present invention for linker molecules to be utilised, which means that time consuming and potentially costly and complex wet chemistry approaches for linkage are not required.

As indicated above the present invention can be utilised to attach functional biological molecules to surfaces of a wide variety of metal, semiconductor, polymer, composite and/or ceramic substrates, which will also be referred to herein simply as “substrates”. For example the substrate may take the form of a block, sheet, film, foil, tube, strand, fibre, piece or particle (eg. a nano- or micro-particle such as a nano- or micro-sphere), powder, shaped article, indented, textured or moulded article or woven fabric or massed fibre pressed into a sheet (for example like paper) of metal, semiconductor, polymer, composite and/or ceramic. The substrate can be a solid mono-material, laminated product, hybrid material or alternatively a coating on any type of base material which can be non-metallic or metallic in nature, and which may include a polymer component, such as homo-polymer, co-polymer or polymer mixture. Indeed, the substrate may also form a component of a device, such as for example a component of a diagnostic kit or detection device, a tissue, cell or organ culture scaffold or support, a biosensor, an analytical plate, an assay component, a micro- or nano-device that interacts with or includes biological components (e.g. molecular motors involving actin/myosin filaments) or a medical device such a contact lens, a stent (eg a cardiovascular or gastrointestinal stent), a pace maker, a hearing aid, a prosthesis, an artificial joint, a bone or tissue replacement material, an artificial organ, a heart valve or replacement vessel, a suture, staple, nail, screw, bolt or other device for surgical use or other implantable or biocompatible device.

Other devices that may be produced according to the invention are those related to chemical processing. For example, the invention includes devices utilised in chemical processes conducted on surfaces or substrates that may result in generation of fuels, biofuels, electricity or production of chemical products (e.g. bulk or fine chemicals, drugs, proteins, peptides, nucleic acids, polymers, food supplements and the like). In a preferred embodiment the invention includes devices used in the production of ethanol by the action of enzymes on sugars or cellulose or other agents. The invention also includes devices used in production of electricity by means of a chemical reaction catalysed by an enzyme, such as in a fuel cell or bio-fuel cell and fuel cells or substrates that incorporate a biological processing component (e.g. fuel cells comprising photosynthetic cells). The functionalised metal, semiconductor, polymer, composite and/or ceramic can for example form an electrode of such a fuel cell. In this context the invention provides surfaces functionalised by enzymes that can be made available to chemical agents to be processed by immersion in them or by arranging for the agents to flow over the surfaces. In the case that the agent flows over the enzyme-functionalised surface, problems with the poisoning of the enzyme by the products of the reaction can be minimised. Another advantage of the invention is that the enzyme functionalised surface can be rapidly and conveniently replaced with another fresh functionalised surface in the event that the enzymes become poisoned or are otherwise rendered inactive, without the need to dispose of the entire batch of chemicals.

A further specific example of devices of the invention is semiconductors, such as CMOS devices, that can be used for the detection of biological molecules by sensing the specific attachment of the target molecules to detection molecules bound on the semiconductor surface, or that are components of bio-devices including bio-computers (for example involving proteins, peptide or nucleic acids).

Throughout this specification the term “plasma polymer” is intended to encompass a material produced on a surface by deposition from a plasma, into which carbon or carbon containing molecular species are released. The carbon containing molecular species are fragmented in the plasma and a plasma polymer coating is formed on surfaces exposed to the plasma. This coating contains carbon in a non-crystalline form together with other elements from the carbon containing molecular species or other species co-released into the plasma. The surface may be heated or biased electrically during deposition. Such materials often contain unsatisfied bonds due to their amorphous nature.

The term “hydrophilic” refers to a surface that can be wetted by polar liquids such as water, and include surfaces having both strongly and mildly hydrophilic wetting properties. For a smooth surface we use the term hydrophilic to mean a surface with water contact angles in the range from 0 to around 90 degrees. The most preferable water contact angle for the hydrophilic surfaces relating to the present invention are in the range of around 50 to about 70 degrees.

As a result of the plasma treatment according to the invention under plasma immersion ion implantation (PIII) and/or co-deposition and/or plasma polymer surface deposition conditions the present inventors have determined that not only is the substrate surface activated to allow binding of one or more biological molecules, but that the possibly hydrophobic nature of the surface is modified to exhibit a more hydrophilic character. This is important for maintaining the conformation and therefore functionality of many biological molecules, the outer regions of which are often hydrophilic in nature due to the generally aqueous environment of biological systems. The inventors have also shown that not only do techniques of the present invention give rise to hydrophilicity of the treated metal, semiconductor, polymer, composite and/or ceramic surfaces, but that as a result of cross linked sub-surface regions in the plasma polymer there is a delay to the hydrophobic recovery of the surface that takes place over time following the treatment, relative to polymer surfaces that are plasma treated but without energetic ion bombardment conditions. The inventors understand that the mechanism associated with delayed hydrophobic recovery is that in addition to the treatment giving rise to surface activation it also results in improved surface stabilisation. This stabilisation is understood to result from penetration into the sub-surface of the coating by energetic ions, giving rise to regions of cross-linking in the plasma polymer sub-surface. Although the surface is likely to be rough on an atomic scale, meaning that it is difficult to define the surface as a smooth plane, the energies of ions utilised will ensure that they penetrate at least about 0.5 nm into the interior of the deposited plasma polymer and up to about 500 nm from the growth surface during deposition. It is therefore intended for the term “sub-surface” to encompass a region of the plasma polymer, which may be the entire interior of the plasma polymer layer or part thereof, subject to plasma deposition under energetic ion bombardment conditions, that is between about 0.5 nm and about 1000 nm beneath the final coating surface, preferably between about 5 nm and about 500 nm, 300 nm or 200 nm, and most preferably between about 10 nm and about 100 nm beneath the surface.

The term “polymer” as it is used herein is intended to encompass homo-polymers, co-polymers, polymer containing materials, polymer mixtures or blends, such as with other polymers. The term “polymer” encompasses thermoset and/or thermoplastic materials, as well as polymers generated by plasma deposition processes. The term “polymer” also encompasses polymer like surfaces that include reactive species or electrons and which may approach, generally or in isolated regions, the appearance and structure of amorphous carbon. The polymer surfaces may fully or partially coat or cover the substrate, may include gaps or apertures and/or regions of varied thickness, where the gaps or apertures and regions of varied thickness may be consistent, ordered, patterned and/or repeated or may be random or disordered.

The plasma polymer surface created in the process can be generated through plasma ion implantation with carbon containing species, co-deposition under conditions in which substrate material is deposited with carbon containing species while gradually reducing substrate material proportion and increasing carbon containing species proportion and/or deposition of a plasma polymer surface layer with energetic ion bombardment. In this context the carbon containing species may comprise charged carbon atoms or other simple carbon containing molecules such as carbon dioxide, carbon monoxide, carbon tetrafluoride or optionally substituted branched or straight chain C₁ to C₁₂ alkane, alkene, alkyne or aryl compounds as well as compounds more conventionally thought of in polymer chemistry as monomer units for the generation of polymer compounds, such as n-hexane, allylamine, acetylene, ethylene, methane and ethanol. Additional suitable compounds may be drawn from the following non-exhaustive list: butane, propane, pentane, heptane, octane, cyclohexane, cycleoctane, dicyclopentadiene, cyclobutane, tetramethylaniline, methylcyclohexane and ethylcyclohexane, tricyclodecane, propene, allene, pentene, benzene, hexene, octene, cyclohexene, cycloheptene, butadiene, isobutylene, di-para-xylylene, propylene, methylcyclohexane, toluene, p-xylene, m-xylene, o-xylene, styrene, phenol, chlorphenol, chlorbenzene, fluorbenzene, bromphenol, ethylene glycol, diethlyene glycol, dimethyl ether, 2,4,6-trimethyl m-phenylenediamine, furan, thiophene, aniline, pyridine, benzylamine, pyrrole, propionitrole, acrylonitrile, pyrrolidine, butylamine, morpholine, tetrahydrofurane, dimethylformamide, dimethylsulfoxide, glycidyl methacrylate, acrylic acid, ethylene oxide, propylene oxide, ethanol, propanol, methanol, hexanol, acetone, formic acid, acetic acid, tetrafluormethane, fluorethylene, chloroform, tetrachlormethane, trichloilnethane, trifluormethane, vinyliden chloride, vinyliden fluoride, hexamethyldisiloxane, triethylsiloxane, dioxane, perfluoro-octane, fluorocyclobutane, octafluorocyclobutane, vinyltriethoxysilane, octafluorotoluene, tetrafluoromethane, hexamethyldisiloxane, heptadecafluoro-1-decene, tetramethyldisilazane, decamethyl-cyclopentasiloxane, perfluoro(methylcyclohexane), 2-chloro-p-xylene.

In one aspect the plasma polymer surface has a thickness of from about 0.3 nm to about 1000 nm, from about Inn to about 500 nm, 300 nm or 100 nm or from about 10 nm to about 30 nm.

The terms “metal” or “metallic” as used herein to refer to elements, alloys or mixtures which exhibit or which exhibit at least in part metallic bonding. Preferred metals according to the invention include elemental iron, copper, zinc, lead, aluminium, titanium, gold, platinum, silver, cobalt, chromium, vanadium, tantalum, nickel, magnesium, manganese, molybdenum tungsten and alloys and mixtures thereof. Particularly preferred metal alloys according to the invention include cobalt chrome, nickel titanium, titanium vanadium aluminium and stainless steel.

The term “ceramic” as it is used herein is intended to encompass materials having a crystalline or at least partially crystalline structure formed essentially from inorganic and non-metallic compounds. They are generally formed from a molten mass that solidifies on cooling or are formed and either simultaneously or subsequently matured (sintered) by heating. Clay, glass, cement and porcelain products all fall within the category of ceramics and classes of ceramics include, for example, oxides, silicates, silicides, nitrides, carbides and phosphates. Particularly preferred ceramic compounds include magnesium oxide, aluminium oxide, hydroxyapatite, titanium nitride, titanium carbide, aluminium nitride, silicon oxide, zinc oxide and indium tin oxide.

The term “semiconductor” as it is used herein to refers to materials having higher resistivity than a conductor but lower resistivity than a resistor; that is, they demonstrate a band gap that can be usefully exploited in electrical and electronic applications such as in diodes, transistors, and integrated circuits. Examples of semiconductor materials include silicon, germanium, gallium arsenide, indium antimonide, diamond, amorphous carbon and amorphous silicon.

“Composite” materials comprehended by the present invention include those that are combinations or mixtures of other materials, such as composite metallic/ceramic materials (referred to as “cermets”) and composites of polymeric material including some metallic, ceramic or semiconductor content, components or elements. Such composites may comprise intimate mixtures of materials of different type or may comprises ordered, arrays or layers or defined elements of different materials.

The term “polymer” as it is used herein is intended to encompass homo-polymers, co-polymers, polymer containing materials, polymer mixtures or blends, such as with other polymers and/or natural and synthetic rubbers, as well as polymer matrix composites, on their own, or alternatively as an integral and surface located component of a multi-layer laminated sandwich comprising other materials e.g. polymers, metals or ceramics (including glass), or a coating (including a partial coating) on any type of substrate material. The term “polymer” encompasses thermoset and/or thermoplastic materials as well as polymers generated by plasma deposition processes.

The polymeric substrates which can be treated according to the present invention include, but are not limited to, polyolefins such as low density polyethylene (LDPE), polypropylene (PP), high density polyethylene (HDPE), ultra high molecular weight polyethylene (UHMWPE), blends of polyolefins with other polymers or rubbers; polyethers, such as polyoxymethylene (Acetal); polyamides, such as poly(hexamethylene adipamide) (Nylon 66); polyimides; polycarbonates; halogenated polymers, such as polyvinylidenefluoride (PVDF), polytetra-fluoroethylene (PTFE) (Teflon™), fluorinated ethylene-propylene copolymer (FEP), and polyvinyl chloride (PVC); aromatic polymers, such as polystyrene (PS); ketone polymers such as polyetheretherketone (PEEK); methacrylate polymers, such as polymethylmethacrylate (PMMA); polyesters, such as polyethylene terephthalate (PET); and copolymers, such as ABS and ethylene propylene diene mixture (EPDM). Preferred polymers include polyethylene, PEEK and polystyrene.

The term “co-deposition” as used herein refers to a deposition process which deposits at least two species on a surface simultaneously, which may involve varying over time the proportions of the two or more components to achieve graded layers of surface deposition. Most preferably the deposition of this graded layer is commenced with deposition of only the substrate material, noting that layers deposited prior to the deposition of carbon containing species become the effective substrate.

By the term “mixed or graded interface” it is intended to denote a region in the material in which the relative proportions of two or more constituent components vary gradually according to a given profile. One method by which this mixed or graded interface is generated is by ion implantation. This achieves a transition from substrate material to deposited plasma polymer material. During the process any one of, or any combination of, the voltage, pulse length, frequency and duty cycle of the PHI pulses applied to the substrate may vary in time thereby varying the extent to which the species arising from the plasma are implanted. Another example method by which a graded metal/plasma polymer interface can be achieved is co-deposition, where the power supplied to the magnetron or cathodic arc source of metal, or the composition of the gases supplied to the process chamber are varied so that the deposited and/or implanted material changes progressively from more metallic to more polymeric.

The term “plasma” or “gas plasma” is used generally to describe the state of ionised vapour. A plasma consists of charged ions, molecules or molecular fragments (positive or negative), negatively charged electrons, and neutral species. As known in the art, a plasma may be generated by combustion, flames, physical shock, or preferably, by electrical discharge, such as a corona or glow discharge. In radiofrequency (RF) discharge, a substrate to be treated is placed in a vacuum chamber and vapour at low pressure is bled into the system. An electromagnetic field generated by a capacitive or inductive RF electrode is used to ionise the vapour. Free electrons in the vapour absorb energy from the electromagnetic field and ionise vapour molecules, in turn producing more electrons.

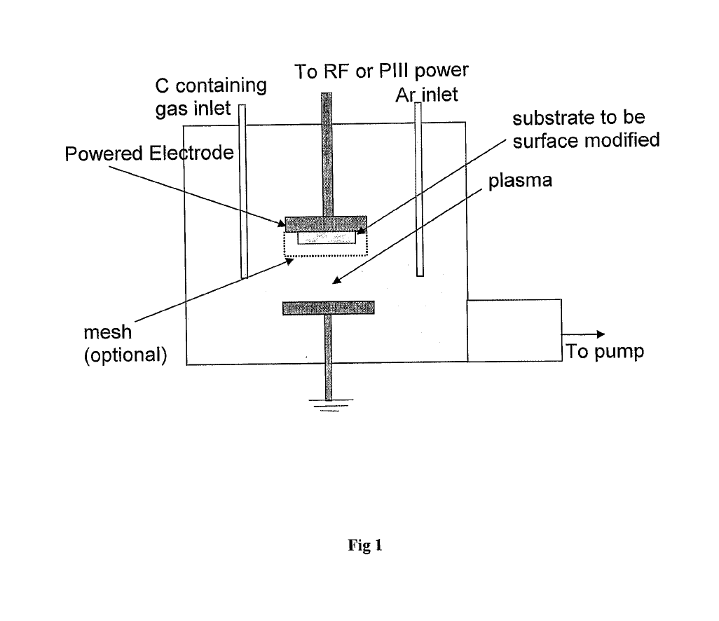

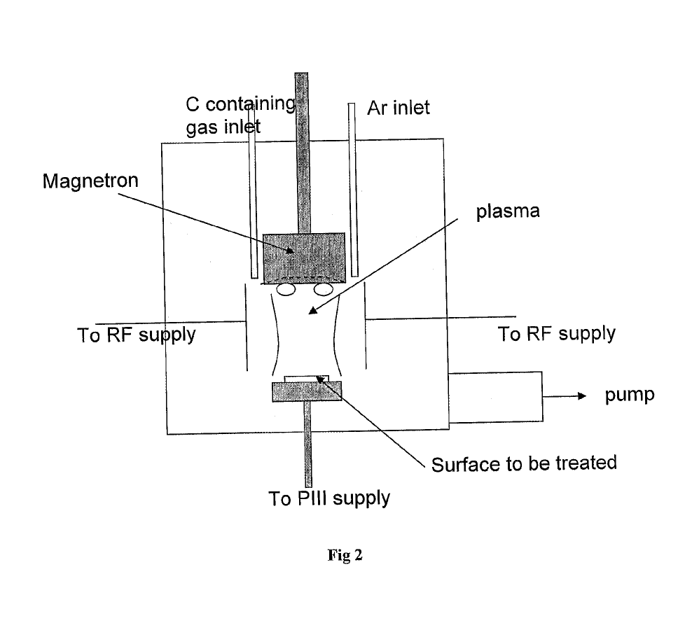

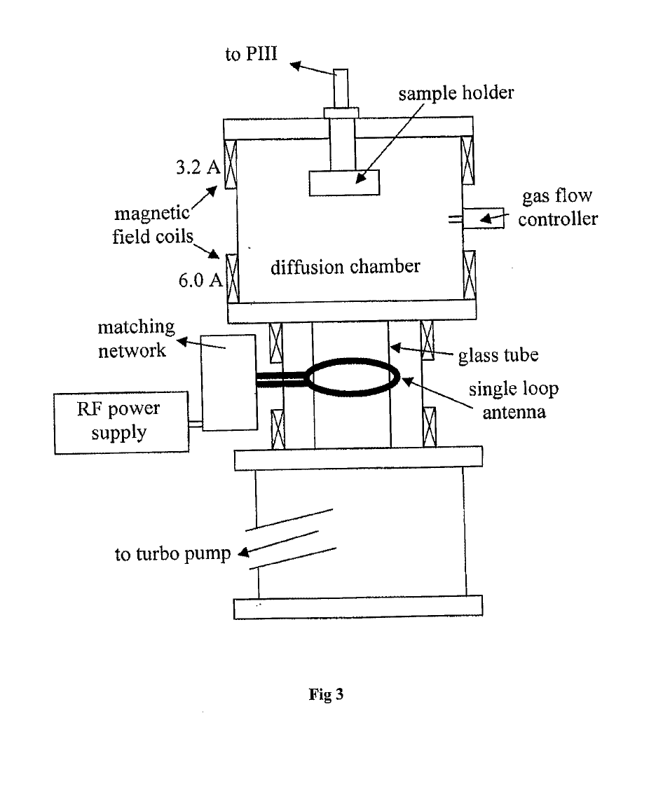

In conducting the plasma treatment according to the invention, typically a plasma treatment apparatus (such as one incorporating a Helicon, parallel plate or hollow cathode plasma source or other inductively or capacitively coupled plasma source, such as shown in FIGS. 1, 2, 3 and 11) is evacuated by attaching a vacuum nozzle to a vacuum pump. A suitable plasma forming vapour generated from a vapour, liquid or solid source is bled into the evacuated apparatus through a gas inlet until the desired vapour pressure in the chamber and differential across the chamber is obtained. An RF electromagnetic field is generated within the apparatus by applying current of the desired frequency to the electrodes from an RF generator. Ionisation of the vapour in the apparatus is induced by the electromagnetic field, and the resulting plasma modifies the metal, semiconductor, polymer, composite and/or ceramic substrate surface subjected to the treatment process.

In one embodiment of the invention it is possible to treat the plasma polymer surface either while it is being deposited or after its deposition, with a plasma forming vapour to thereby activate the plasma polymer surface for binding to biological molecules. Suitable plasma forming vapours used to treat the plasma polymer surface of the metal, semiconductor, polymer, composite and/or ceramic substrate include inorganic and/or organic gases/vapours. Inorganic gases are exemplified by helium, argon, nitrogen, neon, water vapour, nitrous oxide, nitrogen dioxide, oxygen, air, ammonia, carbon monoxide, carbon dioxide, hydrogen, chlorine, hydrogen chloride, bromine cyanide, sulfur dioxide, hydrogen sulfide, xenon, krypton, and the like. Organic gases are exemplified by methane, ethylene, n-hexane, benzene, formic acid, acetylene, pyridine, gases of organosilane, allylamine compounds and organopolysiloxane compounds, fluorocarbon and chlorofluorocarbon compounds and the like. In addition, the gas may be a vaporised organic material, such as an ethylenic monomer to be plasma polymerised or deposited on the surface. These gases may be used either singly or as a mixture of two more, according to need. Preferred plasma forming gases according to the present invention are argon and organic precursor vapours as well as inorganic vapours consisting of the same or similar species as found in the substrate.

Typical plasma treatment conditions (which are quoted here with reference to the power that may be required to treat a surface of 100 square centimetres, but which can be scaled according to the size of the system) may include power levels from about 1 watt to about 1000 watts, preferably between about 5 watts to about 500 watts, most preferably between about 30 watts to about 300 watts (an example of a suitable power is forward power of 100 watts and reverse power of 12 watts); frequency of about 1 kHz to 100 MHz, preferably about 15 kHz to about 50 MHz, more preferably from about 1 MHz to about 20 MHz (an example of a suitable frequency is about 13.5 MHz); axial plasma confining magnetic field strength of between about 0 G (that is, it is not essential for an axial magnetic field to be applied) to about 100 G, preferably between about 20 G to about 80 G, most preferably between about 40 G to about 60 G (an example of a suitable axial magnetic field strength is about 50 G); exposure times of about 5 seconds to 12 hours, preferably about 1 minute to 2 hours, more preferably between about 5 minutes and about 20 minutes (an example of a suitable exposure time is about 13 minutes); gas/vapour pressures of about 0.0001 to about 10 torr, preferably between about 0.0005 torr to about 0.1 torr, most preferably between about 0.001 torr and about 0.01 torr (an example of a suitable pressure is about 0.002 torr); and a gas flow rate of about 1 to about 2000 cm³/min.

According to the present invention the plasma treatment may be under plasma immersion ion implantation (PIII) conditions, with the intention of implanting the sub-surface of the metal, semiconductor, polymer, composite and/or ceramic substrate with the organic carbon containing species. Typical PIII conditions include a substrate bias voltage to accelerate ions from the plasma into the treated substrate of between about 0.1 kV to about 150 kV, preferably between about 0.5 kV to about 100 kV, most preferably between about 1 kV to about 20 kV (an example of a suitable voltage is about 10 kV); frequency of between about 0.1 Hz to about 1 MHz, preferably between about 1 Hz to about 1000 Hz, most preferably between about 100 Hz to about 8000 Hz (an example of a suitable frequency is about 1000 Hz); pulse-length of between about 1 μs to about 1 ms, preferably between about 50 μs to about 500 μs (an example of a suitable pulse-length is about 50 μs).

Following activation of the metal, semiconductor, polymer, composite and/or ceramic substrate surface it is possible to functionalise the plasma polymer surface with a biological molecule or linker by simple incubation (eg. by bathing, washing, stamping, printing or spraying the surface) of the activated surface (substrate) with a solution comprising the biological molecule or linker. Preferably the solution is an aqueous solution (eg. saline), that preferably includes a buffer system compatible with maintaining the biological function of the molecule, such as for example a phosphate or Tris buffer. It may then be appropriate to conduct one or more washing steps also using a biologically compatible solution or liquid, for example the same aqueous buffered solution as for the incubation (but which does not include the biological molecule), to remove any non-specifically bound material from the surface, before the functionalised plasma polymer substrate is ready to be put to its intended use. In another embodiment it is possible to use an agent such as bovine serum albumin (BSA) that will inhibit non-specific adsorption of further biological molecules.

The inventors have determined that both the activated metal, semiconductor, polymer, composite and/or ceramic substrates and the substrates functionalised with biological molecules according to the invention exhibit extensive shelf life. For example, the activated polymer coated substrate may be stored (preferably in a sealed environment) for a period of minutes, hours, days, weeks months or years before incubation with a biological molecule to result in functionalisation of the plasma polymer surface. To the extent that de-activation takes place over time, this can be reversed by annealing, as discussed above. Similarly the substrates functionalised with biological molecules according to the invention may be stored (preferably following freeze drying and more preferably in a sealed environment at low temperature) for periods of minutes, hours, days, weeks, months or years without significant degradation before being re-hydrated, if necessary, and put to their intended use. If freeze drying is adopted a stabiliser such as sucrose may beneficially be added before the freeze drying process. The sealed environment is preferably in the presence of a desiccant and may comprise a container or vessel (preferably under vacuum or reduced oxygen atmosphere) or may for example comprise a polymer, foil and/or laminate package that is preferably vacuum packed. Preferably the sealed environment is sterile to thus prevent or at least minimise the presence of agents such as proteases and nucleases that may be detrimental to activity of the biological molecules. Alternatively the activated or functionalised substrates may be stored in a conventional buffer solution, such as mentioned above.

The invention will now be described further, and by way of example only, with reference to the following non-limiting examples.

### EXAMPLES

### Example 1

### Plasma Treatment of Metal, Semiconductor, Polymer, Composite and Ceramic Substrates for Enhanced Binding of Functional Horseradish Peroxidase

### Materials and Methods

FIG. 1 shows a schematic of the plasma treatment chamber. The source region consists of two parallel electrodes. Radio frequency power at 13.56 MHz or high voltage pulsed power is coupled to the electrodes by a Comdel CPM-2000 matching network or ANSTO PI3 power supply, respectively. The sample is mounted on the powered electrode the other electrode is connected to earth. The base pressure of the chamber is around 3×10⁻⁶ torr.

Acetylene and argon were admitted to the chamber at flow rates of 1.5 sccm and 5 sccm respectively, to a pressure of 150 mT. The unit sccm indicates a flow unit of one standard cubic centimetre per minute. The pulsed power supply is connected and the technique of Plasma Immersion Ion Implantation and Deposition (PIII&D) is used with conditions of 1.5 kV, 10,000 Hz at a 10 μs pulse length. Substrate samples are treated using these conditions for a duration of 20 mins to implant carbon containing species into the metal. Metal substrates of stainless steel, titanium and aluminium were treated.

The high voltage pulsed power supply connection is replaced with the rf power source. Argon and n-hexane gases are then injected into the vacuum chamber at flow rates of 2.5 sccm to a pressure in the chamber of around 2 Pa. The forward power used in the plasma chamber was 100 W, matched with a reverse power of 12 W. The substrate self bias was measured to be −220V. Deposition of a plasma polymer was carried out for 1 min 15 sec. Two examples of the process of the invention are provided, as follows:

### Method (1)

This method describes the production of a covalently bound biological molecule on the surface of a metal or other substrate. The substrate is connected to a pulsed bias power supply capable of delivering 20 kV pulses of typical pulse duration 20 microseconds. A carbon containing gas such as methane, acetylene or n-hexane is introduced into an argon plasma created by a parallel plate capacitor to which an RF field is applied. The bias pulses are applied during the operation of the RF plasma and ions from the carbon containing gas are implanted into the metal to produce a graded interface. The process is completed by depositing a more or less pure plasma polymer from n-hexane.

### Method (2)

This method also describes the production of a covalently bound biological molecule on the surface of a metal or other substrate. The substrate is similarly connected to a pulsed bias power supply capable of delivering 2-20 kV pulses at a pulse duration of 20-500 microseconds. In this case, however, the system is additionally fitted with a sputtering source or a cathodic arc source typically of the same material from which the substrate is made. The sputtering source is typically a magnetron source, either de or rf, balanced or unbalanced. The cathodic arc source can be pulsed or continuously operating.

If a sputter source is used, the source is initially operated in argon with the application of a pulsed bias voltage to the substrate. Ions from the plasma of the sputter source are accelerated the substrate surface having the effect of cleaning the surface and implanting metal into the surface under the influence of the bombarding argon ions. As the deposition and implantation proceeds, metal atoms bombard the surface and grow more material onto the surface while implanting some material below the surface. The precursor gas, n-hexane, is then progressively introduced and the duty cycle and/or voltage of the pulsed bias is progressively reduced. This leads to a graded deposition of metal and plasma polymer with the fraction of metal decreasing. The flow rate of the n-hexane is increased further until the surface of the sputter cathode is poisoned and only pure plasma polymer is deposited. The top surface is the functional surface.

If a cathodic arc source is used, the arc is operated simultaneously with the pulsed power substrate bias initially into a vacuum to achieve plasma implantation and deposition. As the implantation/deposition proceeds, the pressure of the precursor gas is increased, so that a composite of the plasma polymer and the metal is deposited and implanted. At the end of the deposition/implantation, a pure or almost pure deposition of a plasma polymer is achieved, which produces the functional surface.

For both of the above implementations of method (2) an additional plasma generating source to assist in breaking down the plasma polymer precursor can be added. For example, this may take the form of a capacitively coupled rf discharge in the vicinity of the surface to be functionalized.

After treatment by method (1) and/or method (2), samples are incubated with the protein horseradish peroxidase (HRP). The HRP is from Sigma, P6782. A 10 mM phosphate (PO₄), pH 7 buffer is used. Unless otherwise stated, the HRP concentration in the buffer solution is 50 ug·ml⁻¹. The protein concentration is verified by absorption from the Heme group at 403 nm using the extinction coefficient of 102 mM·cm⁻¹ [1].

After overnight incubation in the HRP buffer solution, samples are washed 6 times for 20 minutes in fresh buffer solution. Untreated samples are used as controls. After washing, each sample is clamped between two stainless steel plates separated by an O ring (inner diameter 8 mm, outer diameter 11 mm) which is sealed to the plasma treated sample surface. The top plate contains a 5 mm diameter hole, enabling the addition of 75 μl TMB (3,3′,5,5′ tetramethylbenzidine, Sigma T0440), an HRP substrate, to an area of polymer surface determined by the diameter of the O ring. After 30 secs, 50 μl aliquots are taken and added to 50 μl of 2 M HCl, in a 100 μl cuvette to stop the reaction. The optical density (O.D.) at a wavelength of 450 nm is measured in transmission through the cuvette using a DUO 530 Life Science UV/VIS spectrophotometer. Each data point is the average of measurements taken from at least 3 samples.

To determine relative estimates of the amount of protein (functional or not before and after washing with SDS detergent) left on the surface, infrared spectra are obtained using a Digilab FTS7000 FTIR spectrometer. The spectra are taken in attenuated total reflectance (ATR) mode using a multiple bounce germanium crystal, at a resolution of 1 cm⁻¹.

### Results

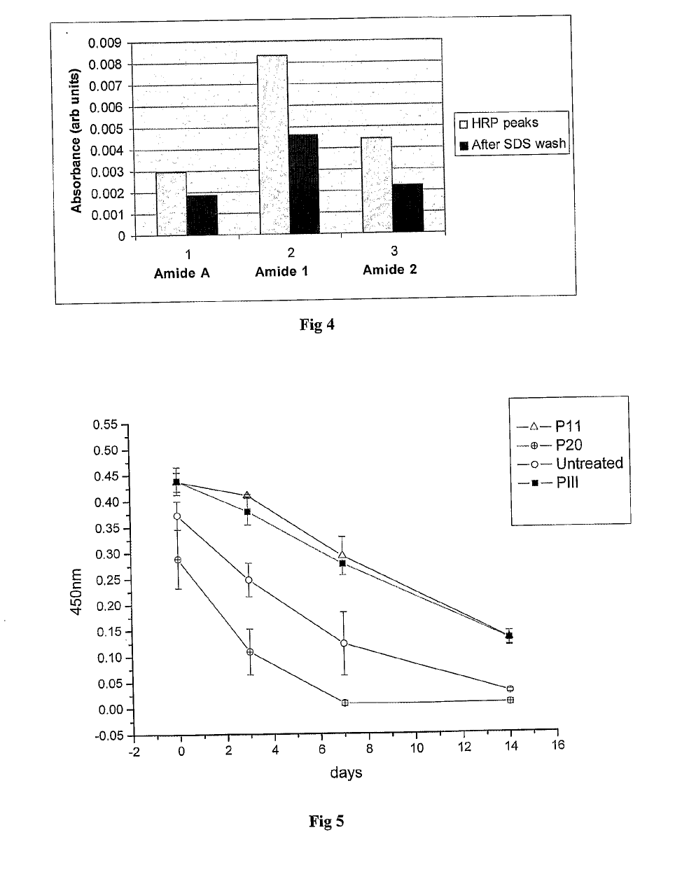

Two samples were deposited: one (P11) with the substrate placed on the powered electrode which had self bias of −220 V on it and another (P20) with the substrate placed on the grounded electrode and not subject to self bias. After incubation in HRP solution overnight both samples were tested for covalent attachment by washing in SDS. SDS solution is capable of removing molecules that are not covalently bound. The data shows that a significant proportion of the HRP remains and is therefore covalently attached to the surface. Evidence of covalent attachment of the enzyme horse radish peroxide for sample P11 is shown in FIG. 4. Protein was still present on sample P11 as shown by the presence of amide peaks in the FTIR spectrum. The height of the peak indicates the amount of protein present. No amide peaks were detected after SDS washing for sample P20.

FIG. 5 shows retained protein function for sample P11 and P20. The absorbance at 450 nm indicates that the protein is biologically active and retains a high degree of the activity over the measurement period with regular washing in fresh buffer. Comparison surfaces are a PIII treated polyethylene sheet and untreated polyethylene sheet. Sample P11 retained the protein function as well as the PIII treated polyethylene and much better than both the untreated polyethylene and sample P20.

### Discussion

Our interpretation of the mechanisms for enhanced functional protein attachment occurring on the surface of the plasma polymerised layer of the substrate is as follows. The highly defective surface contains reactive sites that covalently bind the proteins in solution. This type of binding is robust enough to resist repeated washing cycles. The deposited plasma polymer layers also have hydrophilic surfaces so that surface induced denaturing of the protein is reduced. Ion implantation and/or deposition into the surface prior to or during the deposition of the plasma polymer is used to create a gradual graded transition to the underlying material and therefore a very strong interface to the plasma polymer layer.

### Example 2

### Plasma Treatment of Substrates for Enhanced Binding of Functional Catalase

### Materials and Methods

The materials and methods adopted are the same as for Example 1, but with the exception that instead of HRP, plasma treated polymer surfaces are incubated with the enzyme catalase (Sigma cat. no. C3155). An assay using surface exposure to hydrogen peroxide containing solution is then conducted according to the method of Cohen et al ², as hydrogen peroxide is consumed in a reaction catalysed by catalase, to determine catalase functionality. The surface is incubated with 6 mM H₂O₂ and allowed to react for 6 minutes on an ELISA plate shaker, before an aliquot is taken and measured for remaining hydrogen peroxide. The remaining H₂O₂ is measured by adding excess ferrous ions, which are converted to ferric ions. Ferric ions are then reacted with thiocyanate to form a reddish/orange coloured complex which absorbs at a wavelength of 475 nm. The optical density at this wavelength thus provides a measure of the quantity of H₂O₂ remaining.

When optical density is measured the optical density of a 6 mM solution of hydrogen peroxide control solution is also measured.

### Results and Discussion

Catalase functional binding to the treated surfaces is expected to be greater than for non-treated surfaces. The functional binding is similar for surfaces treated with a simple RF discharge and for those treated also with PIII. However, activity of bound catalase is expected to be maintained at a higher level over the course of the experiment in the case of polymer surface treated to generate cross-linked sub-surface regions by energetic ion bombardment conditions. It is believed that plasma treatment under energetic ion bombardment conditions is more effective than simple plasma polymer deposition treatment of a polymer in maintaining biological molecule functionality due to the cross-linked sub-surface slowing of the rate of hydrophobic recovery of the plasma polymer surface. The mixed or graded interface generated between the plasma polymer and the underlying metal, semiconductor, polymer, composite and/or ceramic substrate is important for ensuring that the functionalised plasma polymer layer is not removed due to contact with solution or biological environment or due to mechanical stress.

### Example 3

### Effect of Tween 20 on Functional Attachment of Catalase to Plasma Treated Substrate

### Materials and Methods

Catalase (Bovine liver catalase (EC 1.11.1.6) (C-3155, 20 mg/ml)) is attached to two sets of activated substrate surfaces using the same approach as for Example 2. One set of surfaces is treated with 10 mM PO₄ 0.005% Tween 20 (from BDH) for one hour whereas the other set is not treated with Tween 20. Catalase in 10 mM PO₄, 0.005% Tween 20 pH 7 is then added to both sets of surfaces and incubated overnight with rocking. Samples are then washed as in Example 1 with 10 mM PO₄ pH 7 buffer. No Tween 20 is included in the washing steps.

### Results and Discussion

Detergents have long been used in ELISA assays for blocking areas of plasma polymer surface not coated with bound antigen and for washing off loosely bound antigens, antibodies and reagents. In particular, non-ionic Tween 20 detergent has been widely used because it permanently blocks a surface and does not appear to affect the function of the protein under assay. The blocking action is expected to be almost complete for untreated surfaces and plasma treated surfaces. The same result of strong blocking is expected whether the surface is blocked first with Tween, or if Tween and catalase are added simultaneously. To confirm that the effect is a blocking of attachment and not an inhibition of protein function, Tween 20 is added to catalase in solution and is expected to have no adverse effect on the function of the enzyme. The experiment is also carried out in 10 mM PO₄ containing 0.15M NaCl at pH 7 and also in PBS buffer at pH 7.4 with and without added Tween 20. In both cases Tween 20 is expected to inhibit functional attachment to all surfaces.

### Example 4

### Effect of Sodium Chloride on Functional Attachment of Catalase to Plasma Treated Substrate

### Materials and Methods

Catalase (Bovine liver catalase (EC 1.11.1.6) (C-3155, 20 mg/ml)) is attached to activated substrate surfaces using the same approach as for Example 2. Catalase is incubated in solutions of different NaCl concentrations overnight and washed as in Example 1, but in a solution of the same NaCl concentration that the protein is soaked in and where for the sixth wash the samples are transferred to new falcon tubes and all samples are washed in 10 mM PO₄.

### Results and Discussion

Electrostatic interactions between proteins and between proteins and surfaces are screened by the presence of ions in solution. To determine the role of electrostatic forces on the surface-protein interaction, we consider the effect of NaCl concentration on the attachment of catalase. We expect that increasing salt concentration will not reduce, but rather, increase the amount of functional activity on all of the surfaces. This implies that either more protein is attached or that the attached protein is better dispersed on the surface so its functional sites are more accessible. Catalase is known to aggregate in solution and perhaps higher salt concentrations could dissociate aggregates, resulting in a higher enzyme activity with the same amount of protein. Binding not being reduced in the presence of salt is indicative that the interactions responsible for a large fraction of the binding are not of an electrostatic nature (ie. not based on charges and/or interactions between permanent dipoles). This would be consistent with covalent binding of catalase to the activated substrate surface.

### Example 5

### Effect of Surface Ageing on Functional Attachment of Catalase to Plasma Treated Substrates

### Materials and Methods

Catalase (Bovine liver catalase (EC 1.11.1.6) (C-3155, 20 mg/ml)) is attached to activated substrate surfaces using the same approach as for Example 2. Before conducting the catalase functional assay as in Example 2 the activated polyethylene samples are stored at room temperature for 4 months in a plastic container that is not airtight.

### Results and Discussion

Results for the stored treated surfaces are expected to be identical to samples that have catalase attached immediately after treatment. These results would show that the plasma treatment is stable for at least 4 months.

### Example 6

### Stability Over Time of Functional Horseradish Peroxidase Attached to Plasma Treated Substrate

### Materials and Methods

Substrate surfaces are exposed to plasma treatment and to incubation with HRP and activity assay under same conditions as for Example 1.

To assess the short term stability of the attached protein over time treated substrate samples are kept in buffer solution which was replaced with fresh buffer each day. The assay is carried out on samples removed from the solution on the day following incubation (day 0), the day after that (day 1) and then every other day (days 3 and 5).

To assess the longer term stability (shelf life) of the treated surfaces, the above procedure is repeated with surfaces that are stored in a desiccator in dry air at room temperature and atmospheric pressure for 2, 4 weeks, 6 months and 1 year periods prior to incubation in the protein solution.

### Results and Discussion

The results are expected to demonstrate that any aging effect in treated samples is very small and has stabilised after 2 weeks.

### Example 7

### Examination of Mechanism of Binding of Soybean Peroxidase to Plasma Polymer Surfaces

### Materials and Methods

Pieces of substrate are cut into small samples approximately 1 cm×1 cm in size. These samples are then cleaned with methanol and transferred into the plasma treatment chamber for treatment under the conditions outlined in Example 1. All protein attachment experiments are carried out on untreated control samples for comparison.

Phosphate buffer (PB) is 10 mM NaH₂PO₄ and 10 mM Na₂HPO₄, pH 7.0. Standard phosphate-buffered saline (PBS) is PB containing 150 mM NaCl adjusted to pH 7.4. Seed coat Soybean Peroxidase (SBP) is from Sigma-Aldrich and is chosen because its activity on a surface is easily determined by the use of a colorimetric assay. In the assay the reaction of a SBP substrate, 3,3′,5,5′-tetramethylbenzidine (TMB) is stopped with acid, forming a yellow reaction product, the optical density of which is read at 450 nm. Unlike horseradish peroxidase (HRP), SBP exists in only one isoform, and generally has greater stability.

Lyophilized SBP is reconstituted into buffer. The extinction coefficient ε403=94.6 mM-1 cm-1 is then used to calculate the protein concentration⁴. The protein is then diluted with buffer to the concentrations used in the experiments.

After treatment, the samples and the untreated controls are incubated overnight in a solution of buffer containing SBP added to a concentration of 50 μg mL⁻¹ unless otherwise stated. The samples are then transferred to a new container and washed six times in fresh buffer solution, resting on a rocker for a period of 20 min for each wash. The samples are then stored in a tube in fresh buffer until they were measured using the TMB assay. If the samples are to be stored for longer periods, the solution is replaced with fresh buffer daily. The samples selected to be assayed on a given day are placed in small holders which consist of two metal layers with a 7 mm diameter hole in the centre of one layer surrounded by a O-ring to seal the liquid in. 75 μL TMB is allowed to react for 30 sec, after which 50 μL is removed and acidified for spectrophotometry at 450 nm. The absorbance measured is related to the amount of functional protein on the surface. To determine relative estimates of the amount of protein (functional or not) left on the surface, infrared spectra are obtained using a Digilab FTS7000 FTIR spectrometer. The spectra are taken in attenuated total reflectance (ATR) mode using a multiple bounce germanium crystal, at a resolution of 1 cm⁻¹.

### Results and Discussion

It is expected that the treated surfaces will show much greater retention of active protein over the 10 day period compared to the untreated control.

### Example 8

### Freeze Drying Horseradish Peroxidase on Substrate

### Material and Methods

Substrate surfaces are exposed to plasma treatment according to the methods outlined in Example 1. Both plasma treated and untreated surfaces are incubated overnight in horseradish peroxidase (50 ug/ml) in 10 mM phosphate buffer pH7. Next day the samples are washed in 10 mM phosphate buffer pH 7 six times, 20 minutes each time. To the last wash we add sucrose to a final concentration of 2.5%. The solution is then frozen with the samples in a 500 ml round bottom flask or in a 50 ml falcon tube by immersing the container in liquid nitrogen. When frozen the water is removed by attaching the round bottom flask to a Dynavac FD 1 freeze dryer. Falcon tubes are placed inside the freeze dryer. We then freeze dry overnight. Freeze drying is a process in which the aqueous content of the materials is removed by sublimation into a vacuum. A successful freeze drying step will enable the function of the attached molecule to be restored upon rehydration. After freeze drying overnight the samples are removed and placed in a sealed container with desiccant and stored at 4° C. Samples are stored with desiccant and then rehydrated and exposed to the HRP activity assay as in Example 1 at selected time points following freeze drying.

### Results and Discussion

We expect that significant activity of HRP bound to the substrate surface will be retained for the surface that is exposed to plasma-treatment.

### Example 9

### Covalent Attachment and Bioactivity of Horseradish Peroxidase on Plasma Polymerised Hexane Coatings

### Materials and Methods

FIG. 1 shows a schematic diagram of the plasma polymerization chamber used. The chamber was pumped using diffusion and rotary pumps, achieving base pressures below 5·10⁻⁴ Pa. Plasma polymerization was performed in an n-Hexane/argon gas mixture and in pure (≧99%) n-Hexane. The precursor, n-Hexane, was purchased from Sigma-Aldrich (cat. No. 139386). The discharge was created using a capacitively coupled electrode powered by a r.f. generator (Ceasar Dressler, 13.56 MHz) through a match box. The powered electrode (81 mm in diameter) was placed 40 mm below the grounded electrode. The working gas mixture pressure was 2 Pa and the total gas flow rate was 5 sccm (2.5 sccm for each component).

Polyethylene sheet substrates (Goodfellow, LDPE ET311452 thickness 0.5 mm) were placed on the powered electrode. The power was adjusted in the range 20-100 W to achieve a negative self bias on the powered electrode, that varied from −80V to −225V (Table 1). As the deposition rate increased with power, the deposition time was adjusted to maintain as close as practical to a constant thickness of the plasma polymer coating. A coating was also prepared without the influence of the negative self bias by depositing onto a substrate held on the grounded electrode. After each deposition the sample was left in the chamber under vacuum for more than half an hour before removal from the chamber to ambient atmosphere.

TABLE 1
List of samples deposited onto polyethylene substrates. Deposition
conditions and O/C ratios determined by XPS as well as the coating
thickness and water contact angle (WCA) are shown. The flow rates
for both Ar and n-hexane were 2.5 sccm and the pressure during
deposition was 2 Pa in all cases. The sample marked * was prepared
with the polyethylene substrate attached to the grounded electrode
with a negative self bias of −190 V appearing on the driven electrode.

| | Power | Bias | | | XPS | | |
|---|---|---|---|---|---|---|---|
| Sample | (Watt) | (V) | Time | Thickness | C(%) | O(%) | WCA |
| PE1 | 20 | −90 | 5 min | 50 nm | 92 | 8 | 84 |
| PE2 | 40 | −155 | 2.5 min | 43 nm | 93 | 7 | 80 |
| PE3 | 60 | −170 | 2 min | 42 nm | 93 | 7 | 86 |
| PE4 | 80 | −215 | 1 min, | 38 nm | 94 | 6 | 85 |
| | | | 15 s | | | | |
| PE5 | 100 | −220 | 55 s | 35 nm | 94 | 6 | 84 |
| PE6* | 60 | 0 | 6 min | 42 nm | 89 | 11 | 90 |

The thickness of the plasma polymer coatings was measured by AFM (Quesant Q-scope 350). The wettability was measured by the sessile water drop method. High resolution XPS analysis of carbon and oxygen 1 s peaks was performed to determine the O/C ratio on the surfaces of the films. This measurement was performed by means of an X-ray photoelectron spectrometer equipped with Al K^{α} X-ray source (1486.6 eV, Specs) and a hemispherical energy analyser (Phobios 100, Specs).

To facilitate structure analysis in the absence of a background of polyethylene absorption peaks, the coatings were deposited onto smaller gold coated glass substrates mounted onto the polyethylene substrate ensuring a sharp transition from the gold to the plasma polymer layer to allow good reflection of probing radiation at the interface. These coatings were therefore not suitable for the protein attachment studies because the poor adhesion to the gold layer precludes incubation in protein solution. The FTIR spectra (Bruker Equinox 55) of these samples were measured in reflectance mode at a high angle of incidence 80° using reflectance unit Bruker A518. Because of the strong reflectance of the infrared beam from the gold—plasma polymer coating interface, the reflectance spectra can be viewed as the transmission spectra of the thin plasma polymerized coating. This is known as reflection-absorption infra red spectroscopy (RAIRS).

Protein attachment properties of the surfaces were assessed using the enzyme, horseradish peroxidase (HRP), purchased from Sigma (cat.No.P6782). Samples were incubated in HRP solution (50 μg/ml HRP in 10 mM sodium phosphate buffer pH 7) overnight at 23° C. The incubation time was selected from previous experiments for ultra high molecular weight polyethylene (UHMWPE) and polystyrene (PS) surfaces which showed that the protein absorption saturated after about one hour for PIII treated samples but overnight incubation was needed to saturate adsorption on the untreated polymer surfaces. After incubation, the samples were washed six times (20 minutes each wash) in fresh buffer (10 mM sodium phosphate buffer pH 7). Samples used for FTIR spectroscopy analysis were additionally washed in de-ionised water for 10 seconds to remove buffer salts from the sample surface prior to spectra acquisition.

Bioactivity of the attached HRP was measured by clamping the samples (13 mm×15 mm) between two stainless steel plates separated by an O-ring (inner diameter 8 mm, outer diameter 11 mm) which sealed to the plasma treated surface. The top plate contained a 5 mm diameter hole through which TMB (3,3,5,5′ tetramethylbenzidine, Sigma T0440) was added to the plasma polymer surface. After 30 seconds 25 μl was removed and added to 50 μl of 2M HCl followed by 25 μl of unreacted TMB to make the volume up to 100 μl, The optical density of the solution was then measured at a wavelength of 450 nm using a DU 530 Beckman spectrophotometer.

FTIR-ATR spectra from the films deposited onto polyethylene and used for protein attachment were recorded using a Digilab FTS7000 FTIR spectrometer fitted with an ATR accessory (Harrick, USA) with trapezium Germanium crystal and incidence angle of 45°. To obtain sufficient signal/noise ratio and resolution of spectral bands we used 500 scans and a resolution of 1 cm⁻¹. Before recording spectra, the surface of the samples was dried using dry air flow. Differences, obtained by subtraction, between spectra of samples before and after treatment were used to detect changes associated with the surface treatment and subsequent attachment of protein.

To illuminate the attachment mechanism, samples with attached protein were washed in 2% SDS detergent at 70° C. for 1 hour and then washed with de-ionized water 3 times to remove the residual SDS. FTIR ATR spectra were recorded before and after the SDS treatment and the difference spectra of protein incubated samples and buffer incubated samples determined. All spectral analysis was done using GRAMS software.

### Results and Discussion

### Analysis of FTIR Spectra of Plasma Polymers

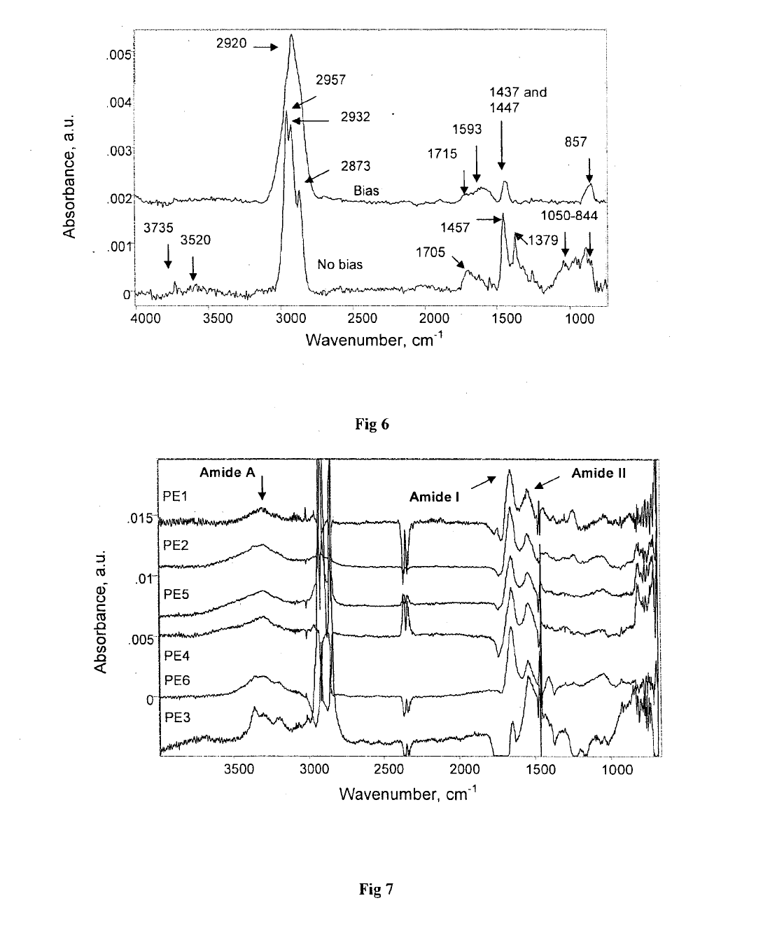

The molecular structure in the coatings was examined by FTIR-RAIR spectroscopy of the coatings polymerized on gold coated glass as satellite samples. Spectra from films polymerized on grounded and self biased electrodes are shown in FIG. 6. Table 2 shows the line assignments for the FTIR spectra of the coatings polymerized on the grounded and biased electrodes.

TABLE 2
Spectral line (cm⁻¹) assignments for FTIR spectra of
plasma polymer films deposited on biased and non-biased
electrodes as obtained from the spectra deconvolution.

| Non-biased | Biased | Assignment by |
|---|---|---|
| 3735 | 3735 | ν(OH) |
| 3520 | 3520 | ν(OH) |
| 3033, 3016, | 3070, 3051, 3039, | ν(═CH) |
| 2994 | 3027, 3007, 2987 | |
| 2958 | 2961 | ν(CH₃)ₐₛ |
| 2931 | 2930 | ν(CH₂)ₐₛ |
| 2915, 2899 | 2905 | ν(CH) |
| 2874 | 2879 | ν(CH₃)_{symm} |
| 2849 | 2855 | ν(CH₂)_{symm} |
| 2827 | 2827 | ν(CH₃) |
| — | 2789 | ν(CH₂) |
| 1705 | 1715 | ν(C═O) |
| — | 1593 | ν(C═C) |
| 1457 | — | δ(CH₂), δ(CH₃), |
| — | 1437, 1447 | δ(CH₂) |
| 1379 | — | δ(CH₃), |
| 1050 | — | ν(C—O) |
| 980-844 | 857 | γ(═CH) |

The spectrum for the sample plasma polymerized on the grounded electrode shows strong absorption lines of C—H stretch vibrations at 2957, 2932 and 2873 cm⁻¹ and of C—H bending vibrations at 1457 and 1379 cm⁻¹. These lines are sufficiently narrow to be recognized as hexane molecular fragments. Additional features visible in the spectrum of the film plasma polymerized without substrate bias include: a weak wide line at 3520 cm⁻¹ corresponding to hydroxyl group vibrations; a wide band in the 1750-1600 cm⁻¹ region containing absorptions of the carbonyl C═O and carbon-carbon C═C stretch vibrations; a broad band consisting of a number of individual lines in the 1050-844 cm⁻¹ region, which are attributed to C—O stretch vibrations (around 1050 cm⁻¹) and C—H out-of-plane vibrations in unsaturated carbon structures (around 980-844 cm⁻¹). Unsaturated carbon-carbon bonds appear as a result of hexane dehydration in the plasma discharge. The hydroxyl and carbonyl groups originate from reactions between active free radicals in the plasma polymerized coating and oxygen from the laboratory atmosphere and/or residual gas in the deposition chamber.

The FTIR spectrum of the coating polymerized on the biased electrode is quite different. The lines in the high frequency region (3000-2800 cm⁻¹) are not resolved and the line associated with the C═O vibration is of lower intensity than the C═C vibration line. Since the extinction coefficient is much higher for the C═O vibration line than for the C═C vibration line, the observed difference in these line intensities shows that the concentration of C═O groups is much lower than the concentration of C═C groups in this coating. The 1379 cm⁻¹ line, associated with —CH₃ group bending vibrations, and the lines in the 1050 cm⁻¹ region, attributed to C—O vibrations, are not observed in the film plasma polymerized on the biased electrode.

The lines of the C—H stretch vibrations (2800-3000 cm⁻¹) were used for detailed analysis of the differences in molecular structure between these two coatings. The spectrum of pure hexane contains four lines in this region: 2957 cm⁻¹ due to asymmetrical —CH₃ vibrations, 2931 cm⁻¹ due to asymmetrical —CH₂— vibrations, 2872 cm⁻¹ due to symmetrical —CH₃ vibrations and 2857 cm⁻¹ due to symmetrical —CH₂— vibrations, which are overlaid with each other.

### FTIR ATR Investigation of Protein Attachment

The plasma polymerized hexane coated polyethylene sheets as well as uncoated polyethylene sheets (used as controls) were incubated in HRP containing buffer solution. The samples were then washed in fresh buffer and analyzed for protein activity using a TMB colorimetric assay. The physical presence of bound protein irrespective of its activity was observed by FTIR ATR spectroscopy. Before FTIR ATR spectra acquisition the samples were washed in de-ionized water, to remove residual salts from buffer solution, and subsequently dried.

To detect surface attached protein FTIR ATR spectra were taken from the plasma polymer surfaces before and after incubation in protein containing buffer solution. FIG. 7 shows the difference spectra. Spectral lines attributed to vibrational modes of protein molecules: Amide A at 3300 cm⁻¹, Amide I at 1650 cm⁻¹ and Amide II at 1540 cm⁻¹ are clearly visible. The strength of each line for a given quantity of protein is proportional to its extinction coefficient, εᵢ. The ratios of extinction coefficients, ε(Amide I)/Σ(Amide A)=2.78 and ε(Amide I)/ε(Amide II)=2.13, as previously recorded for a layer of HRP adsorbed on polyethylene, were used to weight the integrated intensities of the Amide A and Amide II lines respectively. In this way, a weighted average of the three lines, equally representative of the signal from all three, could be calculated and used as a relative measure of the amount of protein contributing to the IR adsorptions.

The concentration of attached protein was found to be correlated with the intensity of the spectral lines associated with carbonyl groups as shown in FIG. 8. The total amount of attached protein increases slightly with increasing carbonyl group concentration and then saturates. The figure shows that the attached protein concentration on the plasma polymerized coatings is higher than that on the untreated polyethylene.

### Protein Activity

The results of the TMB colorimetric assay used to measure protein activity over a two week period with daily washing are shown in FIG. 9. Higher bioactivity is observed on the plasma polymerized coatings compared to untreated polyethylene. The activity of protein attached to the plasma polymerized layers shows little variation between samples made with varying bias. In contrast, the activity of the protein attached to the sample coated while held on the grounded electrode is significantly lower and drops at a faster rate than that on the other samples including on the untreated polyethylene. Activity of the HRP on the surface plasma polymerized without bias is reduced to background levels after only 7 days while it takes 14 days to be reduced to the same levels on the untreated polyethylene substrate. All of the plasma polymer layers deposited with bias still show significant enzyme activity after 14 days.

The attachment of protein molecules on surfaces may be due to many different types of interactions. Non-specific physisorption occurs due to a large number of weak dipole-dipole interactions between permanent or induced dipoles on the protein and the polymer surface [9]. A physisorbed protein layer can be removed using detergents, which provide similar or stronger intermolecular interactions with the protein molecules. SDS is a detergent used for protein removal from almost all materials including polymers. It interferes with even the strongest interactions involved in physisorption and as such will remove protein that is not covalently bonded to the surface. The SDS cleaning procedure is widely accepted as a method to test whether proteins are covalently attached to surfaces or not [7, 8].

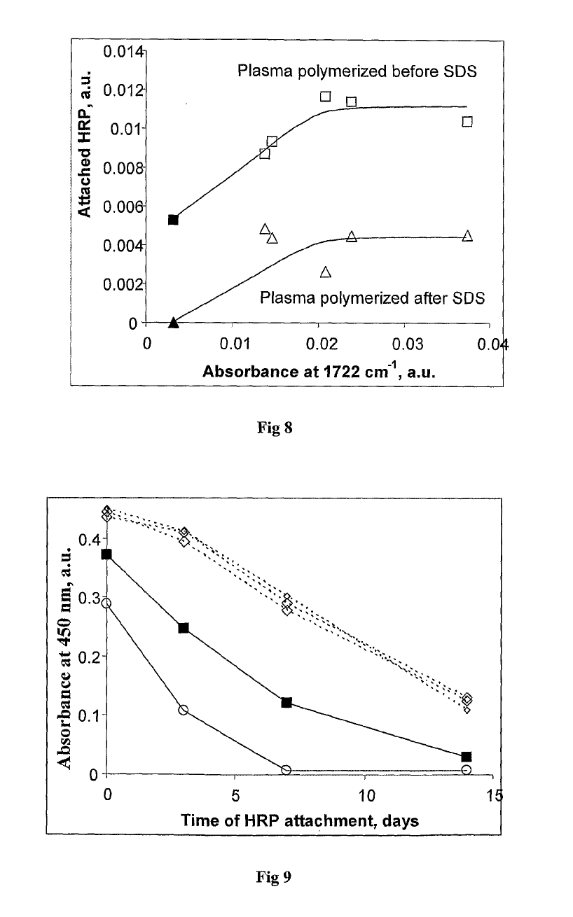

The protein covered samples were washed in SDS detergent and then in de-ionized water. FIG. 10 shows the FTIR ATR spectra recorded from them after drying. The amide lines completely disappeared in the spectra corresponding to the untreated polyethylene surface while they remained, although reduced in intensity, on the plasma polymerized layers deposited on the biased and unbiased electrodes. The weighted averaged intensities of the three protein lines which remain in the spectra of SDS washed samples are shown in FIG. 8. This shows that the amount of protein has decreased twofold on the plasma polymerized samples while it is completely removed from the untreated polyethylene surface. The fact that SDS is known to interfere with non-specific interactions and it successfully removed all protein from the untreated control indicates that the protein remaining on the plasma polymerized coatings is covalently attached.

### Conclusions

Plasma polymerization was used to synthesize a coating from hexane on polyethylene substrates and gold coated glass. The coating was found by FTIR spectroscopy to have a complex structure formed by residual fragments of hexane monomers cross linking and reacting with residual and/or atmospheric oxygen to introduce oxygen-containing groups, unsaturated carbon-carbon groups and crosslinks. Layers that were plasma polymerized under the influence of negative self bias showed structures indicative of higher levels of damage in the residual fragments of the hexane monomers than those synthesized on the grounded electrode.

The enzyme, horse radish peroxidase (HRP) was found to bind covalently to the coatings polymerized on both biased and grounded electrodes, however its bioactivity was maintained significantly better on the layers grown with bias than on those grown on the grounded electrode. The uncoated polyethylene controls showed no evidence of covalent binding as well as poor retention of enzyme bioactivity.

### Example 10

### Plasma Polymerisation Process for Depositing Surfaces Capable of Direct Covalent Enzyme Immobilisation Compatible with CMOS

Complementary metal-oxide-semiconductor (CMOS) refers to both a particular style of digital circuitry design, and the family of processes used to implement that circuitry on integrated circuits. Combining nano-CMOS technology and biosensors will enable miniaturised biological-laboratory-on-chip systems and sensors to be developed. For a reliable sensor, a surface that can bind biomolecules robustly while preserving bioactivity is required. The most robust method for immobilising a biomolecule on a surface is through the use of covalent bonds. The covalent binding sites as well as properties of the surface, such as its interface energy with solution, determine the bioactivity of the attached, molecule and its lifetime. Covalent attachment of enzymes without involving additional chemical linker groups on the functional or sensing probes is preferred to minimise the number of wet chemical processing steps that could compromise compatibility with CMOS processing.

In order to integrate the functional surfaces for immobilizing enzymes or other biological agents with CMOS, a process for their manufacture compatible with CMOS devices is needed. In order to achieve such a compatible process, the surfaces should be smooth and stable both biochemically and mechanically. Furthermore, in CMOS fabrication processes, pattern formation requires layer masking (or lithography). The surface must therefore be etchable, and able to withstand annealing at high temperatures (e.g., 400° C.). Importantly, after all the CMOS processes such as the plasma etching and high temperature annealing, the sensing surfaces on CMOS devices need to retain the enzyme immobilization capability and their bioactivity so that the devices can be used readily to immobilize biological agents during their shelf-life period without introducing complexity of surface treatment.

This example explores the development of biochemically functional and stable surfaces for direct integration into CMOS devices, which can be readily used for covalent immobilization of biological agents (such as enzymes) without the application of additional chemical linkers. The critical CMOS processes for integration of biocompatible surfaces include plasma etching and annealing at approximately 400° C. Enzyme immobilization property and activity are to be studied for comparison after the critical processes.

### Materials and Methods

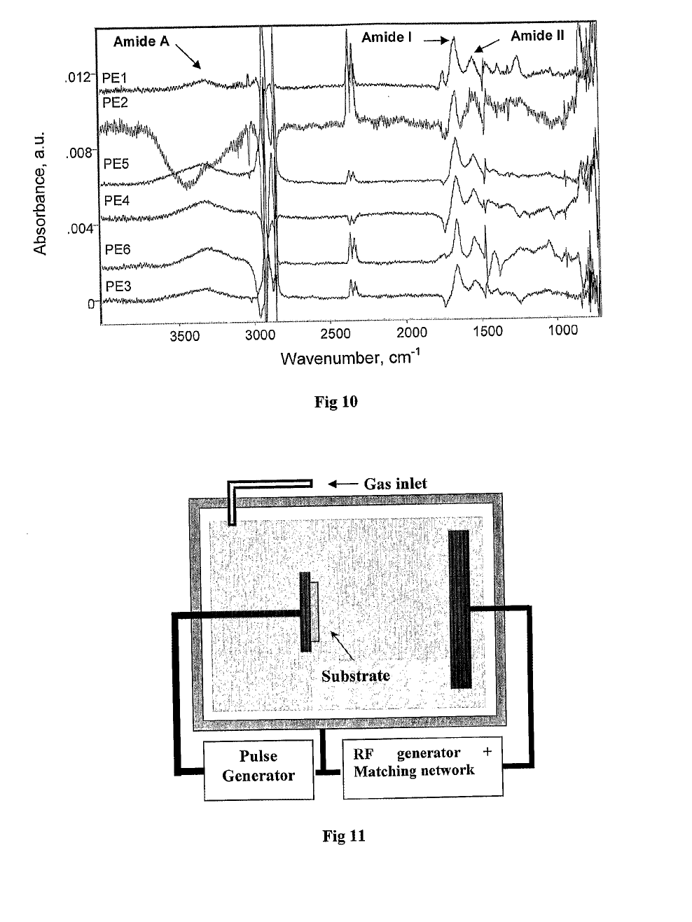

A plasma polymerization method was applied in this work to functionalize silicon or other surfaces compatible with CMOS process. The plasma polymerization process is similar to plasma enhanced chemical vapour deposition (PECVD), a method widely used in the CMOS manufacturing industry. FIG. 11 shows a schematic diagram of the plasma polymerisation system adopted. Specifically, the plasma polymerization system included two plasma sources: one radio frequency (RF) electrode at 13.56 MHz operated at 150 W; the other one was a pulse voltage source with pulse length 10 μs and repetition 10 KHz in this work. Gases injected into the plasma chamber included acetylene, nitrogen, and argon. Gases injected into the plasma chamber were 8 sccm (standard cubic centimetres per minute) acetylene, 4 sccm nitrogen, and 4 sccm argon. Pressure of the system was maintained at 20 Pa, and flow rate was regulated by MKS mass flow controllers. Substrates were placed onto the pulsed electrode, which was immersed into the plasma generated by the RF electrode.

The plasma polymerized surfaces were characterized using a J.A. Woollam M-2000 spectroscopic ellipsometer, attenuated total reflection (ATR) Fourier transform infrared (FTIR) spectra (obtained using a Digilab FTS7000 FTIR spectrometer fitted with an ATR accessory with a traperzium germanium crystal and incidence angle of 45°), and an adhesion tensile tester (Instron 5567). The strength of the surface adhesion was determined by fracturing through tensile stresses at the interface between the substrate and the plasma polymerized layer. Some plasma polymerized samples were further processed by argon plasma etching, or oxygen plasma etching, or annealing in vacuum.

Enzymes tested were all purchased from Sigma without further purification including horseradish peroxidase (HRP, Cat No P6782), soybean peroxidase (SBP, Cat No P1432), and bovine liver catalase (Cat No C3155). The main results presented in this example are from HRP. The procedure for HRP attachment and activity analysis is as follows. Plasma polymerized samples after processes such as as-deposited, plasma etching, and annealing were incubated for 20 hours in HRP (50 μg/ml) in 10 mM phosphate buffer (PB) at pH 7.0. Incubations were in 75 mm sterile Petri dishes with rocking. After the incubation, the samples were washed 6 times each for 20 minutes in 10 mM phosphate buffer pH 7.0. The first wash was performed in the Petri dish used for enzyme incubations. Then the samples were transferred to a clean Petri dish for the next 5 washes. Active HRP on the surfaces of the samples was measured by clamping the sample (approx 15×15 mm) between two stainless plates separated by an O-ring (inner diameter 8.0 mm, outer diameter 11.0 mm) that sealed the surface. The top plate contained a 5.0 mm diameter hole enabling the addition of 75 μl of the HRP enzyme substrate, TMB (3,3′,5,5′ tetramethylbenzidine, Sigma Cat No T0440). After 30 seconds, 25 μl of reacted TMB was taken and added to 50 μl of 2 M hydrochloric acid to stop the reaction. A further 25 μl of unreacted TMB was then added to bring the volume to 100 μl. The absorbance of the solution at 450 nm was then measured using a Beckman DU530 Life Science UV/VIS spectrophotometer. To assess the stability of the attached enzyme with time, samples were kept in buffer for various times after HRP attachment and washing. Buffer was changed every two days so the samples were not affected by bacterial growth in the phosphate buffer. All enzyme activity tests including rocking time were performed at 23±1° C.

Samples from PB solution for analysis in dry environment were rinsed in de-ionized water 5 times, followed by drying in mild nitrogen gas flow, and then placed onto sample stage for analysis. Spectral ellipsometry analysis was conducted using silicon substrate by collecting spectra at three angles of 65, 70, and 75 degrees for conditions before plasma polymerization, after plasma polymerization, and after enzyme attachment respectively. Enzyme attachment was simulated by applying the obtained optical constants of silicon and the plasma polymerized layer. Quartz crystal microbalance with dissipation monitoring (QCM-D) technique was used to measure HRP attached on plasma polymerized quartz crystals. The QCM-D apparatus was from Q-sense (Model E4) and was operated at 25° C. with 5 MHz AT-cut quartz crystals. PB solution was pumped through the QCM-D cell before injecting HRP (in PB) into the cell. HRP attachment was also investigated by other two methods: 1, TMB enzyme activity test after a prolonged rocking rinse (days and up to 3 weeks in this work); and 2, ATR FTIR analysis before and after incubation in sodium dodecyl sulphate (SDS) aquatic solution (2%) by shaking at temperature 70° C. for 1 hour. SDS is a detergent that solubilizes enzyme molecules and is used to remove the enzyme molecules from the substrate surfaces if they are not covalently bonded. Surface contact angle measurements were performed at 23±1° C. using a DSA10-MK2 contact angle analyzer. Sessile water drops of 10 μl were used for advancing contact angle.

### Results and Discussion

Plasma polymerization was applied to functionalize the silicon in a process compatible with CMOS processing. The plasma polymerization process used is often referred to as plasma enhanced chemical vapour deposition (PECVD), a method widely applied in the CMOS manufacturing industry. Adhesion of the plasma polymerized surfaces on silicon was tested using an adhesion tensile tester. The adhesion strength was typically in the range between 18 and 22 MPa. This adhesion strength is sufficient for in vivo medical application, which suggests that any disintegration or mechanical failures of the plasma polymerized surfaces is unlikely during clinical in vivo applications.

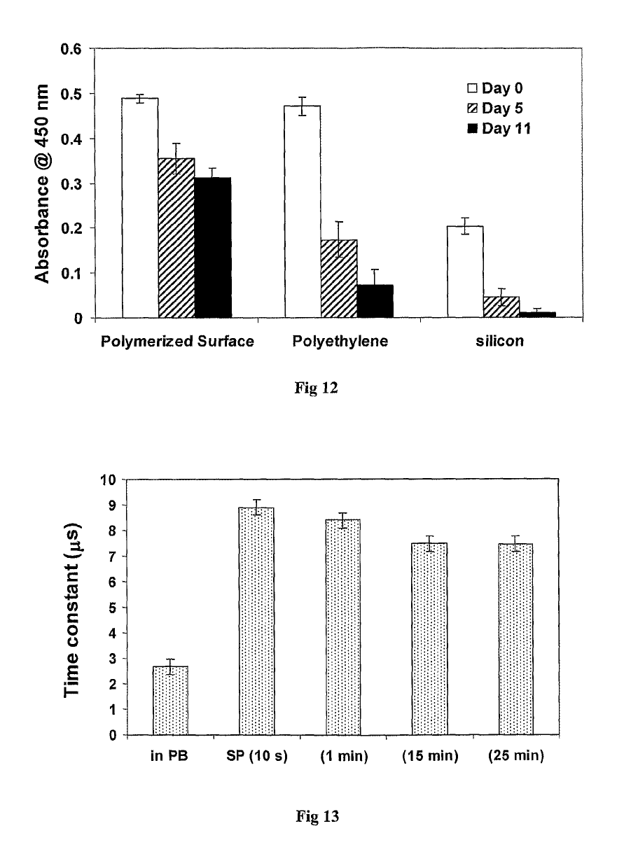

The enzyme activity results are shown in FIG. 12 for the immobilized HRP enzyme activity on the silicon substrate, a polyethylene surface (as a control), and our plasma polymerized surface at different numbers of days after immobilization. At day 0, immediately after enzyme immobilization, the plasma polymerized surface had comparable enzyme activity to that of the polyethylene surface, activity on silicon surface was about 60% lower. After 5 days of washing in PB solution, the activity of the enzymes on the polyethylene surface dropped to about 50% of that on the plasma polymerized surface, while silicon showed an activity of less than 15% of the plasma polymerized surface. By day 11, the activity for the polyethylene surface had fallen to close to 20% of the activity for the plasma polymerized surface, while the enzyme activity on silicon vanished completely. These results show that the plasma polymerised surface retains enzyme activity much better than the other two surfaces.

Sodium dodecyl sulphate (SDS) aquatic solution (2%) was used to attempt removal of the immobilized enzymes from the surfaces by shaking the samples in Falcon tubes containing the SDS solution at a temperature of 70° C. for 1 hour. SDS treatment was conducted after HRP enzyme attachment and attenuated total reflection Fourier transform infrared spectra (ATR FTIR) analysis was done before and after the SDS treatment. To ensure sufficient substrate flexibility for the ATR FTIR measurements the plasma polymerised surfaces used were deposited onto thin stainless steel foil (thickness 25 μm). Polyethylene sheets were used as control surfaces. After SDS washing, the remaining HRP was quantified by the vibration peaks of Amide A, Amide I and Amide II. These peaks indicated that between 75 and 90% of the initial quantity of HRP remained on the plasma polymerized surfaces while they were not detectable on the polyethylene sheets. This suggests that a large proportion of the HRP enzyme molecules are bonded covalently onto the plasma polymerized surfaces.

Atomic force microscopy was used to analyse the roughness of the surfaces deposited onto a polished silicon substrate. The bare silicon surfaces had a RMS roughness of about 0.3 nm. The roughness of the plasma polymer surfaces was about 0.5 to 1 nm, just slightly higher than that of bare silicon. The small roughness of the surfaces enables the surfaces to be processed in nano-CMOS manufacturing processes without introducing additional complexity. Directly after incubation in HRP Enzyme containing solution, the silicon and plasma polymerized surfaces had roughness of 1.8 nm and 1.4 nm respectively. We suggest that the slightly higher roughness on the silicon surface is due to the fact that there is less enzyme coverage. The AFR images showed very high enzyme coverage on the plasma polymerized surface but much less on the silicon surface.

Ellipsometry was conducted after HRP attachment on the surfaces plasma polymerized on silicon substrates. The amount of HRP attached was found to be a monolayer and the enzyme layer thickness was between 4-6 nm, consistent with HRP molecular dimensions. The effective optical index, n, of the HRP layer was about 1.45. Quartz crystal microbalance with dissipation monitoring (QCM-D) was also used to measure HRP attached onto plasma polymerized quartz crystals. The attached HRP on the plasma polymerized surfaces was found to be 300 ng/cm², which indicates that the HRP molecules are fully packed onto the plasma polymerized surface (assuming the footprint of each HRP molecule is 5×5 nm).

Catalase attachment before and after SDS detergent cleaning was also examined using ellipsometry. To improve the accuracy of the ellipsometry analysis, the plasma polymerised surfaces were deposited onto a 160 nm thick silicon nitride interlayer, previously deposited onto the silicon substrate. A thickness of 7.0 nm and an optical index of 1.66 were measured for a catalase layer freshly attached onto the plasma polymerized surface. After SDS incubation, the thickness was 6.0 nm and the optical index was 1.68. The catalase thickness obtained using ellipsometry agrees with the catalase enzyme dimensions of between 5 and 9 nm quoted in the literature, and taken with the optical index indicates that there is approximately one monolayer of the enzyme attached. The fact that the thickness of the catalase layer after SDS cleaning changed so little supports the conclusion that the enzyme is predominantly covalently bonded to the plasma polymerized surface.

The capacitance of surfaces is useful for the design of CMOS chemical sensors. A surface was produced by depositing a 46 nm plasma polymerized layer onto a doped silicon substrate with a silver-coated backside. A potential difference was established by introducing a voltage pulse across a PB solution cell, in which the plasma polymerized surface was used as one electrode and placed at one end of the solution bath and another platinum electrode was placed on the other end of the cell. Two more platinum electrodes were placed in between as reference electrodes. The experimental procedure was as follows. Firstly, the plasma polymerized surface was placed in PB solution (pH=7.0), and after the potential pulse a decay curve giving a RC time constant, where R is the effective resistance and C is the effective capacitance, was established. We used soybean peroxidase for this experiment rather than horseradish peroxidase because HRP is a mixture of isozymes. Soybean peroxidase (SP) contains a single isozyme with an isoelectric point of 4.1 and is negatively charged at pH 7. Soybean peroxidase was injected into the PB solution cell, and new RC time constants were determined at different times after the soybean enzyme immobilization started. The RC time constant as a function of time is shown in FIG. 13.

The large initial increase of the time constant after the injection of the soybean enzyme into the PB solution is due to the large mass to charge ratio of the soybean enzyme molecule and higher viscous coefficient. The reduction of the time constant after initial injection of the soybean enzyme can be interpreted as the immobilization of the soybean enzyme onto the plasma polymer surface. It appears that the monolayer attachment was completed after about 15 minutes. The attachment of the enzyme reduces the capacitance at the surface because the effective thickness of the capacitor increases. A reduction of the plasma treated surface capacitance of approximately 10% would be expected given the thickness of a monolayer soybean enzyme (approximately 4-5 nm). The time dependence of the decay in the time constant measured with soybean peroxidase attachment is consistent with such a change in the surface capacitance.

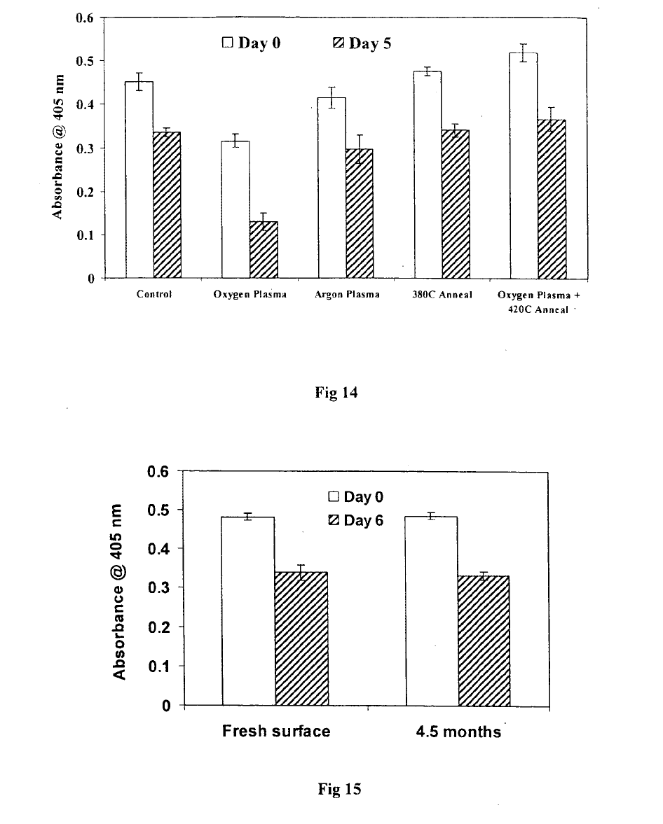

In order to integrate conveniently the plasma polymerised surfaces into CMOS processes, the surface needs to be stable after CMOS compatible plasma etching and annealing, and it must have a long shelf life. The plasma polymerized surfaces can be etched in both oxygen and argon plasma, with oxygen plasma having a much higher plasma etching rate than argon. In FIG. 14 we show the activity of HRP enzyme attached to: an as-prepared plasma polymerized surface (control): such a surface which has been oxygen plasma etched; such a surface which has been argon plasma etched; such a surface after annealing for 30 minutes at 380° C.; and after oxygen plasma etching followed by a 30 minute anneal at 420° C. Activities for day 0 (immediately after attachment and washing in fresh buffer) and day 5 (five days later with daily washing in fresh buffer) are shown for all surfaces. The results indicated that the oxygen and argon plasma etching reduces the activity of the attached enzymes whereas the 380° C. annealing step has no effect on the activity of the attached enzyme. The sample which had been oxygen plasma etched and then annealed at 420° C. showed activity comparable with the as prepared plasma polymerised surface. These results suggest that the plasma polymerized surface can be incorporated into CMOS processing without affecting the activity of the subsequently attached enzymes.

The plasma polymerized surfaces need to have a long shelf lifetime in order to be compatible with CMOS processing. FIG. 15 shows the activity of HRP enzyme layers attached to two samples produced in the same batch (cut from a same plasma polymerized sample piece) but stored in laboratory ambient for different periods of time. The shelf time of the two samples was 1 week (as fresh surface) and 4.5 months respectively. The samples were stored in ambient environment in sealed boxes to prevent dust contamination. The 4.5 month sample was analyzed without further surface cleaning or treatment and had almost the same activity of the attached enzyme layer as the fresh surface for both the day 0 and day 6 measurements after HRP immobilization. There appears to be no sign of degradation with the shelf time, at least up to 4.5 months.

### Conclusions

In summary, plasma polymerized surfaces were produced using the PECVD method, which is widely adopted by CMOS manufacturers. The surfaces produced can covalently immobilise enzymes such as horseradish peroxidase, soybean peroxidase, and catalase. Ellipsometry, QCM-D, AFM, enzyme activity, ATR FTIR, and capacitance measurements indicate that the immobilized enzyme molecules form a monolayer with full packing density. The plasma polymerized surfaces are smooth and strongly adhere to substrates, enabling integration with nano-CMOS devices.

The compatibility of the surfaces with typical CMOS manufacturing processes was investigated. Annealing the as-prepared surfaces does not reduce the activity of the subsequently attached enzyme layer. Plasma etching of the surfaces resulted in degradation of the activity of the subsequently attached enzymes, particularly, in the case of the oxygen plasma etch which is the most convenient method to etch plasma polymerized surfaces in CMOS processing. Fortunately, however, the enzyme activity of the oxygen plasma etched surfaces can be recovered using an annealing process, a step usually applied in CMOS manufacturing at the end of CMOS device formation. The surfaces were found to be stable for enzyme immobilization over the period of shelf time applied in this work without needing additional surface treatment.

### Example 11

### Resistance of Plasma Polymerised Surfaces to Flow Induced Erosion

### Materials and Methods

The plasma polymerization method adopted in this example was as for Example 10, and double-sided polished p-type silicon wafers (100) were used as surfaces to be coated.

The plasma polymerized surfaces were characterized using atomic force microscopy (PicoSPM, Molecular Imaging, Tempe, USA), spectroscopic ellipsometry (J.A. Woollam M-2000), attenuated total reflection Fourier transform infrared spectroscopy (ATR-FTIR, a Digilab FTS7000 FTIR spectrometer fitted with an ATR accessory with a trapezium shaped germanium crystal and incidence angle of 45°, 1 cm⁻¹ spectral resolution, 500 scans), adhesion tensile testing (Instron 5567) and quartz crystal microbalance with dissipation analysis (QCM-D, model Q-sense E4). The durability of the plasma polymerized surfaces under conditions of supra-physiological shear stress was assessed using an in vitro flow circuit with a liquid flow inducer (from Watson Marlow). A segment of the plasma polymerized surface was placed within a short length of 5 mm diameter tubing incorporated within the circuit which was then filled with a mixture of glycerol and de-ionized water (ratio 2:3) to give a solution viscosity much higher than that of human blood in order to accelerate the durability test.

### Results and Discussion

The surface roughness of the acetylene plasma polymerized surfaces was characterized using Atomic Force Microscopy. Typical RMS roughness of the plasma polymer surface was 1-2 nm which is slightly more than the RMS roughness of the bare silicon wafer which was 0.5 nm. The strength of the surface adhesion was determined by separating the interface between the silicon and the plasma polymerized layer in a tensile test. The contact area to the plasma polymerized surfaces was 5 mm in diameter and the measured adhesion strength was typically greater than 20 MPa.

The resistance of the plasma polymerized surfaces to flow induced erosion was analysed using a pulsed flow system. Glycerol mixed with deionised water was used to achieve viscous flow across the plasma polymerized surfaces, which were placed into a 5 mm-diameter pipe in the pulsed flow circuit. In order to accelerate the test, the flow solution was mixed with 60% glycerol in deionised water at temperature 10° C. The pulse frequency was 1.6 Hz and flow rate was 500 ml/min. The resulting shear force on the surfaces was estimated to be equivalent to approximately 10 times the shear force normally encountered in human arteries. The surface was characterized using spectroscopic ellipsometry before and after 3 weeks of continuous flow stressing. No thickness reduction was observed on the plasma polymer surfaces, suggesting very good erosion resistance in cardiovascular applications.

### Conclusions

Acetylene plasma polymerization is a convenient method for creating uniform and smooth plasma polymerized surfaces on a wide range of solid surfaces. It is easily adapted to the deposition of large areas for commercial applications. As a dry plasma process, it lends itself to surface patterning and to deposition onto complex shaped substrates facilitating the production of devices. Tensile testing showed that the adhesion strength of the plasma polymerized layer was at least 20 MPa. This layer was also shown to be resistant to erosion in simulated arterial blood flow conditions.

### Example 12

### Covalent Immobilization of Tropoelastin on Acetylene Plasma Deposited Surfaces with Stainless Steel Substrates

Metallic devices, implants and prostheses typically made from stainless steel and titanium alloys are common place in medicine today so convenient methods of attaching dense active protein layers to metallic surfaces would be beneficial. Applications in cardiovascular surgery and orthopedics in particular rely on the durability, mechanical strength and corrosion resistance of stainless steel. However, the performance of stainless steel is compromised by limited biocompatibility, manifesting as thrombus formation in vascular applications, and eliciting an immune response in wider uses. Previous efforts to improve the biocompatibility of stainless steel have focused on surface properties including smoothness and oxidation, but none have satisfactorily addressed this issue.

In this example surface modification of stainless steel using a plasma deposition process based on plasma enhanced chemical vapour deposition was investigated. The advantages of this method include that it is substrate independent, dry and reliable, creating a smooth and solid organic surface layer. The aim of this work was to develop reliable and ready-to-use surfaces on stainless steel or other non-polymer substrates for covalent immobilization of tropoelastin, a key extracellular matrix protein in blood vessels. The binding of the recombinant human protein tropoelastin forms mature cross-linked elastin by alternating hydrophobic and hydrophilic domains, which confers resilience and elasticity on a diverse range of tissues. The placement of tropoelastin onto metals used for cardiovascular applications requires reliable, strong attachment with sufficient density of immobilized tropoelastin to interact optimally with blood and the relevant cells. Covalent immobilization of tropoelastin is desirable to withstand the shear force of blood flow over long periods.

### Materials and Methods

A schematic diagram of the plasma deposition system is shown in FIG. 11. In this case the substrate is stainless steel. The system includes two plasma producing electrodes: the first is a radio frequency (RF) powered electrode operated at 150 W and 13.56 MHz; the second is an electrode powered by a pulsed dc voltage source with voltage settable between 0 and −1000 V with pulse length 10 us and a repetition rate of 10 KHz. The RF electrode was powered using an ENI-6B RF generator through an ENI-Matchwork matching network. The pulsed dc electrode was driven using an RUP-3 pulse generator from GBS-Elektronik (Germany). Surfaces to be coated were placed on the de pulsed electrode, which was immersed in the plasma generated by the RF electrode. Acetylene was injected into the plasma chamber with a flow rate of 8 SCCM to react and form a polymer-like coating on the exposed surfaces. Nitrogen and argon at various flow rates were also deployed in the gas mix. The plasma gas pressure was maintained at 20 Pa, and the gas flow rates were regulated by MKS mass flow controllers. All experiments were conducted without heating or cooling of substrates and at dc pulse bias of −200 V unless stated otherwise. The resultant substrate temperature during the coating deposition was approximately 30-50° C.

The coated surfaces were characterized using atomic force microscopy (AFM, Autoprobe CP), and spectroscopic ellipsometry (J.A. Woollam M-2000). The surface energy was assessed by sessile drop (10 μl) advancing contact angle measurements, performed at 23±1° C. using a DSA10-MK2 contact angle analyzer. An adhesion tensile tester (Instron 5567) was used to determine adhesion strength of the plasma deposited coatings and the protein layer subsequently attached. For the adhesion strength analysis, the backside of the sample was glued onto a 10 mm diameter disc and the front surface was glued onto a 5 mm diameter disc. The strength of the coating adhesion was determined by applying tensile stress until fracture occurred. A liquid pulse flow inducer (from Watson Marlow) was used to produce a pulsed shear force on the coated surfaces to simulate flow conditions in human artery vessels. The coated surfaces were placed into a 5 mm diameter pipe in the pulsed flow circuit for testing and glycerol mixed with de-ionized water was used as the shearing fluid. The wear caused by the pulsed flow was studied using microscopy and spectroscopic ellipsometry.

For assessment of tropoelastin attachment an ELISA assay was used. Untreated and plasma coated 316L stainless steel foil samples of thickness 25 μm were cut into 1 cm×1 cm squares. A control sample, which was not incubated in the protein containing solution, was tested for each type of surface. The other samples were incubated with 1 ml of 3 mg/ml tropoelastin in PBS at pH 7.4 overnight at 37 C. All of the samples were then blocked with 10 mg/ml bovine serum albumin (BSA) at room temp for 1 h and then washed with PBS before a 1 h at room temperature incubation in 500 μl of monoclonal anti-elastin antibody (primary—Sigma Cat No. E4013 diluted 1:1000 from stock). After washing in fresh PBS, 500 μl of anti-mouse HRP conjugate (secondary—Sigma Cat No. A4416 diluted 1:10,000 from stock) was added for another incubation period. After a further washing step, the samples were transferred to a fresh plate and 500 μl of 2,2′-azino-bis(3-ethylbenzthiazoline-6-sulphonic acid) ABTS solution was supplied to each sample and the absorbance at 405 nm was measured after 1 h at 37° C.

To test for covalent attachment of tropoelastin, samples were incubated in 5% sodium dodecyl sulphate (SDS) in PBS at 90° C. for 10 min. SDS is a detergent that solubilizes protein molecules and removes non-covalently bonded protein molecules.

Horseradish peroxidase (HRP) was attached to plasma coated surfaces and uncoated controls by incubating the samples overnight in 50 ug/ml HRP (Sigma cat no P6782) dissolved in PB buffer. The next day the samples were washed 6 times (for 20 minutes each time) with fresh PB buffer. The attached enzyme activity was measured by clamping the sample (approx, 15×15 mm) between two stainless steel plates separated by an o-ring (inner diameter 8 mm, outer diameter 11 mm) that sealed the surface. The top plate contained a 5 mm diameter hole enabling the addition of 75 ul of the HRP enzyme substrate, TMB (3,3′,5,5′ tetramethylbenzidine, Sigma Cat No T0440). After 30 secs, 25 ul of reacted TMB was taken and added to 50 ul of 2M hydrochloric acid. A further 25 ul of un-reacted TMB was then added to bring the volume to 100 ul. The absorbance of the solution at 450 nm was then measured.

Spectroscopic ellipsometry was used to give complementary information about tropoelastin attachment and coverage. Samples taken from PBS solution were rinsed in de-ionized water 5 times and dried in mild nitrogen gas flow prior to being placed in the spectroscopic ellipsometer for measurement. Spectroscopic ellipsometry data was collected on the plasma coated silicon wafers at three angles of 65°, 70°, and 75° before plasma coating, after plasma coating, and then after tropoelastin attachment. A model was fitted for each data set with the unknown parameters restricted to the top-most layer. The parameters used for the previous layers were imported from models fitted to the preceding data sets.

A quartz crystal microbalance with dissipation analysis (QCM-D, model Q-sense E4) was used to characterize the attachment and de-attachment of tropoelastin. In QCM-D analysis the plasma coated surfaces were placed onto 5 MHz quartz crystals with gold electrodes. The diameter of the quartz crystal was 13 mm with an effective sensing area of diameter 5 mm. All QCM-D analysis was performed at 25° C. and solution was caused to flow over the quartz crystal surface at a rate of 150 ml/min

### Results and Discussion

### Characterization of Plasma Deposited Surfaces

Surface roughness of the acetylene plasma coating deposited on a polished silicon substrate surface was characterized using AFM. The coated surfaces were very smooth. Typical rms roughness was 1-2 nm (c.f. substrate RMS roughness of about 0.5 nm). The water contact angle of plasma coated surfaces was about 62±7°. The adhesion strength as measured by the tensile test method, using a contact area of 5 mm diameter and straining to failure at the coating interface, was typically more than 15 MPa for coatings deposited on stainless steel foils. This value is comparable or larger than the ultimate tensile strength of polyethylene.

The shear strength of the plasma coated surfaces was determined to be very high using a pulsed flow inducer. The liquid pulse flow inducer produced a pulsed shear force on the coated surfaces to simulate the mechanical impact in human blood vessels using a flow of 60% glycerol mixed with de-ionized water at a temperature of 10° C. The pulse flow rate was 500 ml/min with 100 pulses/min, resulting in a shear force on the surfaces equivalent to approximately 10 times the shear force in human artery blood vessels, so as to accelerate the test. No thickness reduction was detected for coatings deposited on polished silicon substrates after 3 weeks continuous flow stressing using spectroscopic ellipsometry. This suggests good mechanical properties for cardiovascular applications.

### Bioactivity of a Surface Attached Enzyme Layer

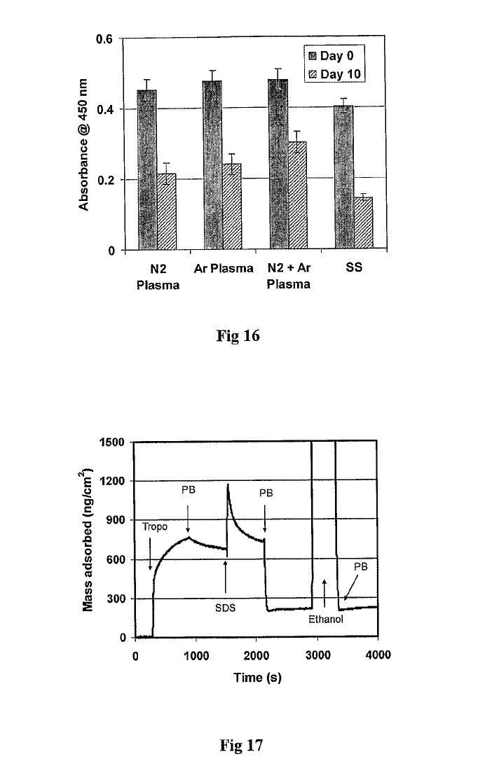

FIG. 16 shows the enzyme activity of horseradish peroxidase for four groups of samples, where the first three groups have differences in the gas mixed with the acetylene precursor while the last group is the untreated control surface. The group denoted as “N2 Plasma” was produced with nitrogen gas flow of 8 SCCM. The group “Ar Plasma” was produced with 8 SCCM argon flow. The group “N2+Ar Plasma” had 4 SCCM flow for each nitrogen and argon; and the group denoted as “SS” was 316L stainless steel foil with no exposure to plasma. All of the plasma coated samples had higher initial activity and much better retention of activity over 10 days compared to the uncoated steel control. Among the three groups of plasma deposited surfaces, the nitrogen mixed with argon acetylene plasma appeared slightly better for maintaining activity to day 10. The N₂/Ar samples were therefore used for tropoelastin attachment and subsequent analysis using QCM-D and spectroscopic ellipsometry.

### Tropoelastin Attachment Analysis Using QCM-D

Tropoelastin is an extra cellular protein which confers strength and elasticity to the skin and other organs in the body. It is used in wound healing and topical skin care (DermaPlus Products) and exists in multiple forms, called polymorphs. In vivo it becomes crosslinked immediately after its synthesis by the cell and during its conversion into the extracellular matrix. Tropoelastin is not normally available in its native state. The method used in this work for obtaining the native state is described elsewhere [10,11].

QCM-D was used to study the dynamics of the attachment of tropoelastin to our plasma deposited surfaces. As tropoelastin attaches to the surfaces coated onto oscillating quartz crystals, a shift in the resonant frequency and a change of dissipation factor (inversely proportional to quality factor Q) are observed. The shift in resonant frequency is proportional to the change in mass associated with the surface attached protein layer. FIG. 17 shows the mass change detected during the surface attachment of tropoelastin and subsequent rinsing with SDS detergent. Initially, the plasma deposited surface was stabilized in 10 mM phosphate buffer (PB) at pH 7.0 for about one hour. Then tropoelastin (concentration 500 μg/ml) in PB buffer was introduced into the flow cell containing the quartz crystal. A rapid mass increase associated with protein attachment to the surface was observed. This was followed by further attachment at a lower rate. After about 10 minutes of exposure to the protein solution, the mass adsorbed was close to 800 ng/cm². The molecular mass of tropoelastin is about 60-70 kDa. Taking the footprint area of tropoelastin molecule in its native state as 30 nm², the monolayer mass should be approximately 350 ng/cm². The much larger adsorbed mass observed suggests the crosslinking of elastin onto a previously attached layer leading to continually increasing coverage in excess of a monolayer. The non-saturating increase of adsorbed mass may be interpreted as multiple tropoelastin molecules crosslinking to form elastin chains or clusters on the surface.

Reverting to flow of fresh buffer resulted in a removal of some of the previously attached mass. Initiation of the flow of SDS detergent appears to give an immediate jump in the apparent mass absorbed. This is an artifact associated with differences in the viscous properties of the SDS and PB solutions and should not be interpreted as an instantaneous change in the adsorbed protein mass. The subsequent steep decrease of the curve however is associated with the removal of surface attached tropoelastin. Once the SDS flow is replaced with PB again it can be seen that the mass of the attached protein layer has been reduced to about 220 ng/cm². An attempt to clean further with 90% ethanol in water did not change the tropoelastin attachment. Once again, the temporary excursion during the ethanol solution flow is a result of its different viscous properties. The mass remaining after SDS and ethanol washing is less than the mass of a monolayer of tropoelastin, suggesting that the SDS resistant layer, which we assume to be covalently bonded to the plasma deposited surface, is “porous” or not fully dense.

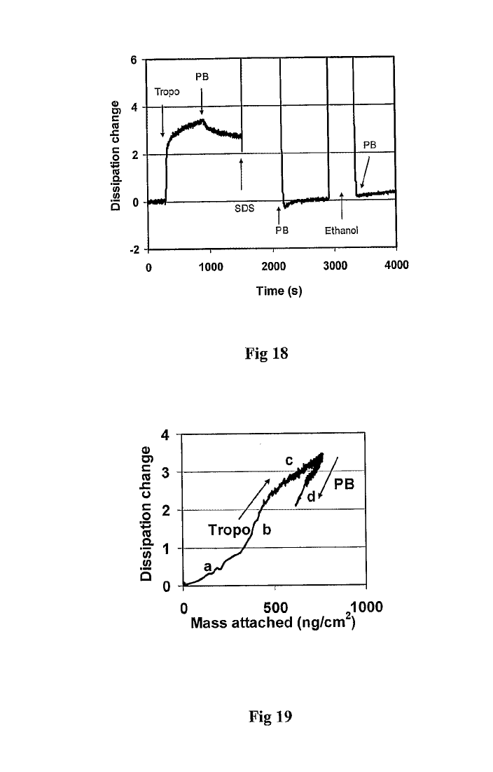

The nature of crosslinked tropoelastin clusters present prior to detergent cleaning is not clear. The changes in the dissipation factor measured in QCM-D provides indications of conformation or attachment density differences between various layers of the bound protein. FIG. 18 shows a plot of the changes in dissipation detected during the QCM-D experiment shown in FIG. 17. The dissipation increased rapidly when the surface was first exposed to tropoelastin containing buffer solution. A slower increase of dissipation, indicating a rougher surface was being created, followed the initial increase. PB washing reduced the dissipation slowly whilst removing some of the attached tropoelastin (as shown in FIG. 17). After SDS cleaning, the dissipation change returned to its original value, although a significant amount of tropoelastin could not be washed away by either the SDS or ethanol.

FIG. 19 shows the dependence of the dissipation change on the attached mass during the tropoelastin soaking and cleaning. The dissipation change during initial tropoelastin soaking to mass about 250 ng/cm² (range: a) was slow, flowed by a faster increase of dissipation (range: b) to the attached mass about 500 ng/cm², then a slower increase of dissipation (range: c). The PB cleaning resulted in a fast reduction of dissipation (range: d). This could suggest that the initial attachment to about 250 ng/cm² is covalently bonded monolayer which is reasonably stiff so that the dissipation increased slowly with mass attached. As tropoelastin attachment continues and polymerized, crosslinked aggregates attach to the initial monolayer, the tropoelastin forms a rough, more porous surface which increases the drag associated with the layer and as a result the dissipation increases more rapidly. With further increases in attached tropoelastin, the roughness stabilizes and the dissipation change becomes slower. PB cleaning causes a rapid reduction in dissipation as the loosely bound tropoelastin on the top of the multilayer stack is washed away and the surface layers become denser and more rigid.

### Optical Analysis

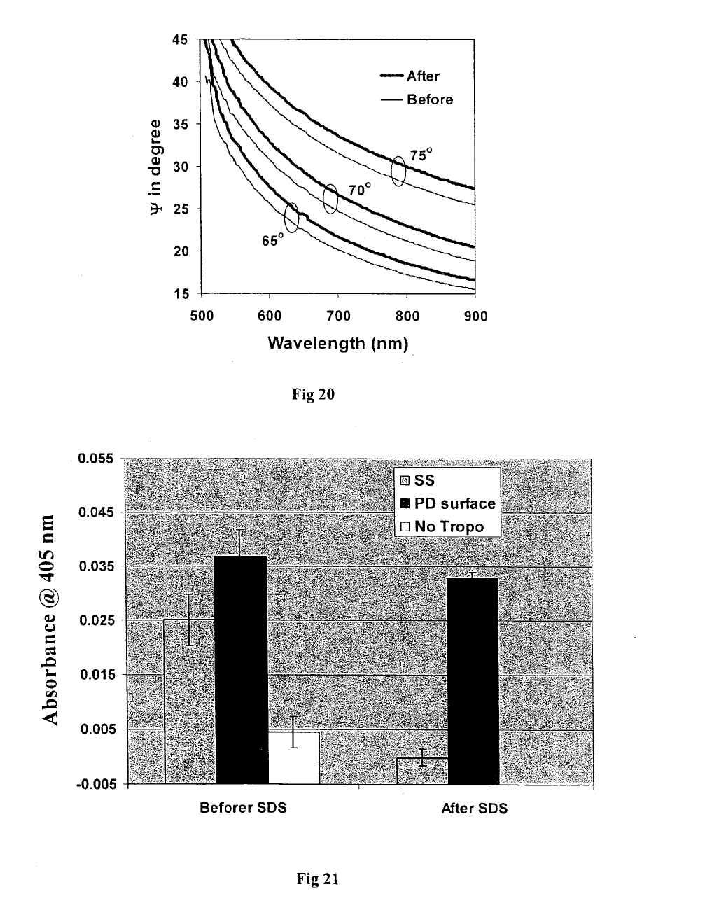

Spectroscopic ellipsometry was used to determine the thickness and optical constants of the attached tropoelastin layer. After incubation in tropoelastin solution and prior ellipsometric analysis, the samples were rinsed 5 times for 10 minutes each time to remove the loosely bound tropoelastin. FIG. 20 shows the raw ellipsometric data obtained from the surfaces before (thin lines) and after (thick lines) incubation in the tropoelastin solution. For improved readability, we offset the 75° and 65° angle data by −5 and 5 degrees respectively. The separation between the thicker lines and the thinner ones indicates that spectroscopic ellipsometry is capable of detecting the attached protein layer.

TABLE 3
Tropoelastin layer thickness and refractive index as a
function of incubation time in tropoelastin solution.

| Incubation time (hours) | 1 | 3 | 24 |
|---|---|---|---|
| Refractive index | 1.32 | 1.43 | 1.52 |
| Thickness (nm) | 4.1 | 6.3 | 5.0 |

The monolayer coverage was analyzed as a function of incubation time using ellipsometry. Table 3 summarizes the simulated thickness and refractive index for incubation times of 1, 3, and 24 hours determined by fitting optical constants and thickness for the tropoelastin layer in a multilayer model including the substrate, the plasma deposited coating and the tropoelastin layer. The data for the underlying layers was determined by ellipsometric analyses on the same sample prior to addition of the tropoelastin layer. The low refractive index obtained after the 1 hour incubation time indicates that the layer may not be a complete monolayer.

### Detection of Surface Attached Tropoelastin by ELISA Assay

FIG. 21 shows tropoelastin attachment, as measured by an enzyme linked immunosorbant assay (ELISA) on untreated and plasma coated stainless steel before and after washing with SDS detergent. A control surface with no tropoelastin bound was included to give an indication of the background signal due to non-specific attachment of the antibody. The coated surface produced a significantly higher absorbance compared with the untreated stainless steel. SDS effectively removes the tropoelastin from the stainless steel surface but not from the plasma deposited surface, indicating a covalent attachment on the latter. The absorbance above background can be interpreted as indicative of the surface concentration of exposed tropoelastin binding motifs recognized by the antibody. The fact that the concentration doesn't change significantly after the SDS washing is consistent with there being about a monolayer of covalently bound protein on the plasma deposited surface. The QCMD result indicates that after protein incubation, tropoelastin is attached in a multilayered structure and that all but the initial monolayer is removed during rinsing and/or SDS washing. Since the antibody used for detection in the ELISA assay would only be able to access motifs in the top layer of protein a multilayered structure, it would be expected to produce a signal corresponding to a monolayer of attached protein both before and after SDS washing as was observed. The fact that the SDS cannot remove a monolayer of tropoelastin indicates that this layer is covalently attached.

FIG. 23 shows high levels of SDS resistant attachment occur for samples deposited with dc pulsed bias ranging from 0 to −1 kV.

### Conclusions

Covalent immobilization of tropoelastin onto metallic materials for implants in medical applications is desirable for achieving surfaces closely identical to biological tissues. The method demonstrated in this work on plasma deposition of organic protein binding coatings onto metallic surfaces provides a convenient approach to convert the issue of covalent immobilization of tropoelastin onto metallic materials into covalent immobilization of tropoelastin onto biocompatible organic surfaces. The advantages of the plasma coating method developed in this work include strong adhesion onto metallic materials, a smooth surface, metal substrate geometry or dimension independence, mechanically strong surfaces, and a dry process. The adhesion strength achieved in this work is compatible to the ultimate tensile strength of some common polymers. It is particularly important that the geometry or dimension independence gives confidence on surface modification of a wide range of irregularly shaped implants such joints, mesh-type stents, and wired flexible supports. HRP activity analysis using a TMB assay showed the plasma polymerized surfaces were significantly better at binding protein layers and maintaining their biological activity than untreated stainless steel.

The plasma deposited surfaces have been demonstrated to immobilize roughly a monolayer of tropoelastin covalently. The covalent interaction of tropoelastin with the plasma deposited surfaces was investigated using quartz crystal microbalance with dissipation analysis, spectroscopic ellipsometry, and ELISA antibody assay. During incubation in tropoelastin, the first layer attached is covalently bonded, while subsequent layers of the protein are physisorbed. The physisorbed layers can be washed away in buffer or SDS detergent. The dissipation analysis suggested that the weakly bonded multilayer contributed to an increase in the dissipation. The spectroscopic ellipsometry and ELISA antibody assay also indicated that the covalently immobilized tropoelastin was in the form of a monolayer.

### Example 13

### Re-Activation of Aged Surfaces by Annealing

### Materials and Methods

Annealing of selected surfaces was carried out by placing the surfaces and heating them in vacuum by thermal contact with a heated surface. The vacuum level in the chamber was 2×10⁻⁴ Pa. The temperature was measured by thermocouple in contact with the heated surface. The annealing temperature was selected between 200° C. to 500° C. The annealing time at the specified temperature was in the range a few minutes to a few hours. Results were obtained for acetylene plasma deposited surfaces on stainless steel substrates RF deposited as described above in Example 12.

After deposition, selected samples were treated in a plasma containing argon with oxygen added a flow rate of approximately 1 sccm. The HRP activity of annealed surfaces was measured after incubation and at 5 days after incubation. The polar and dispersic components of the surface energy of the surfaces were determined by the sessile drop method using two fluids, namely water and formamide. A Kruss contact angle analyser DSA10-MK2 was used to measure the contact angles. The dispersic and polar components of the surfaces energy were calculated from the contact angles. The number of electron spins in the surfaces was measured using an electron spin resonance analyser. For this measurement the samples were deposited onto polyimide sheet of dimensions 50×50 mm. Polyimide was chosen as substrate as it is temperature stable and gives a low ESR signal. It is also suitable for insertion into the ESR cavity.

### Results and Discussion

The results shown in FIG. 14 demonstrate that the oxygen etching of a plasma deposited reduces the amount of functional binding. However, annealing in vacuum at 380 and 420° C. restores the functional binding to levels comparable to those before the oxygen plasma treatment.

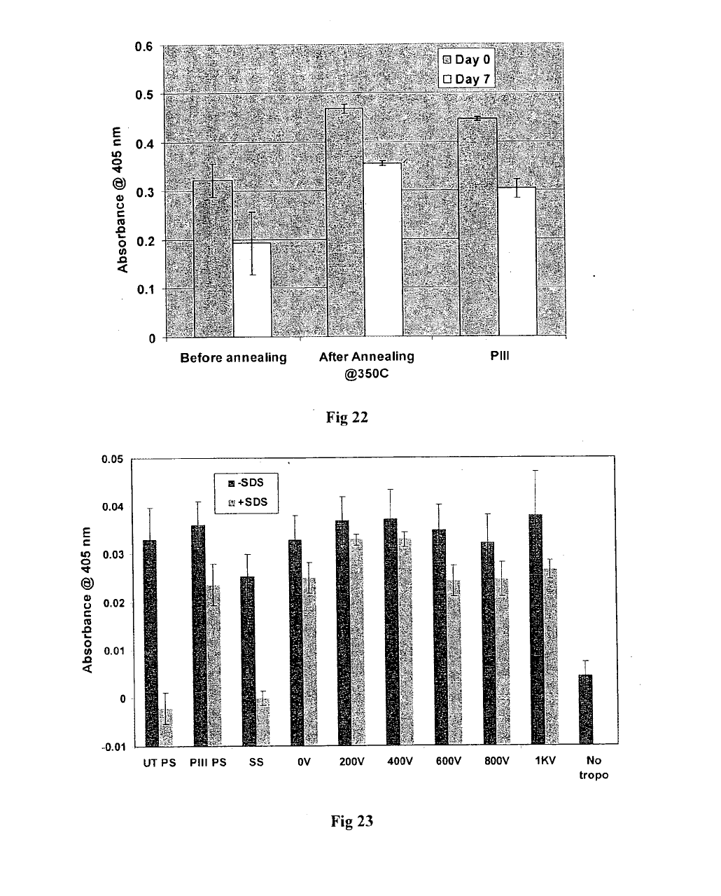

FIG. 22 shows the HRP functional activity immediately after incubation and after 7 days in buffer solution of a acetylene plasma deposited surfaces with and without vacuum annealing at 350 C. A Pill treated polyethylene surface was used as a control. The vacuum annealed surface had the highest activity at both Day 0 and Day 7 and showed the smallest loss of activity.

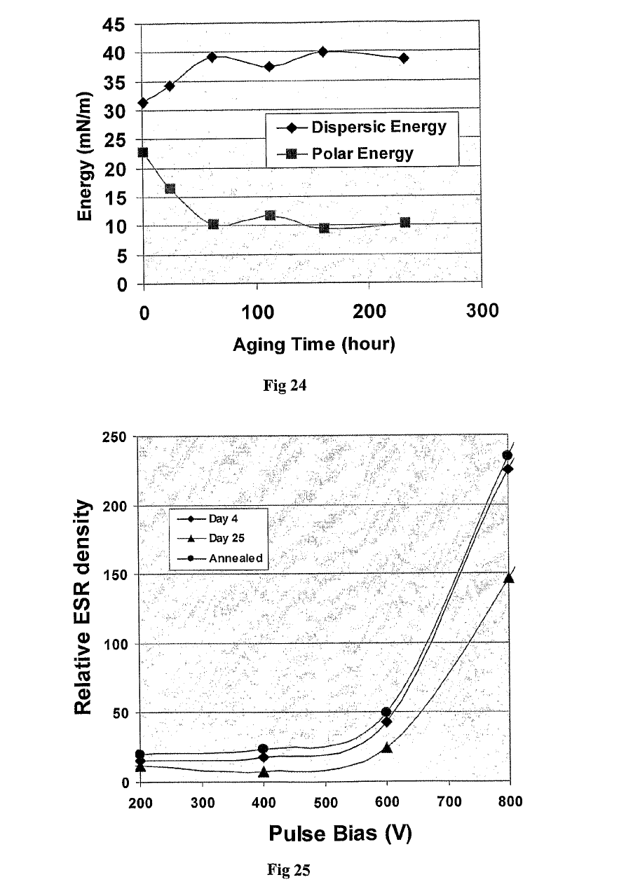

FIG. 24 shows the dispersic and polar components of the surface energy as a function of time in laboratory ambient air. The polar component shows a strong decrease over the first few days, stabilizing at approximately 10 mNm⁻¹. The dispersic component increases slightly and stabilizes at approximately 40 mNm⁻¹

The effect of annealing on both components of the surface energy for the aged samples is shown in Table 4. The annealing has largely restored the polar and dispersic components to their initial value before aging.

Unpaired electrons which are measured as electron spins in ESR are believed to have a role in increasing the polar component of the surface energy as well as playing a role in the covalent bonding of proteins to the surfaces. The effect of vacuum annealing on the number of electron spins in acetylene plasma deposited surfaces is shown in FIG. 25. The number of electron spins depends on the bias voltage used in the preparation of the surfaces as shown in the Figure. For all bias voltages the number of electron spins decreases over time. Annealing restores the original number of spins in the samples.

TABLE 4
The effect of annealing at 350 C. for 20 minutes in vacuum for acetylene
plasma deposited surfaces. The samples were deposited under bias
conditions and aged in air as specified in the table. The dispersic
energy increased from an intial value of 30-35 mN/m to 36-40 mN/m,
and the polar energy decreased from an initial value of 15-20 mN/m
to 8-12 mN/m. Annealing the aged samples recovered both the dispersic
and polar energies close to the initial values.

| Sample age (month) | 6.0 | 5.0 | 7.0 |
|---|---|---|---|
| Bias (V) | 0.0 | 200.0 | 400.0 |
| Dispersic energy before anneal (mN/m) | 36.9 | 39.4 | 37.1 |
| Dispersic energy after anneal (mN/m) | 34.0 | 30.3 | 31.3 |
| Polar energy before anneal (mN/m) | 11.5 | 10.6 | 9.1 |
| Polar energy after anneal (mN/m) | 15.0 | 20.4 | 15.1 |

### Conclusions

Vacuum annealing is effective in restoring the polar component of the surface energy as well as the number of electron spins in plasma deposited surfaces. The annealed surfaces also show increased retention of the function of the attached protein. These results show that an annealing step is therefore useful to increase the effective life of plasma deposited surfaces.

### Example 14

### Deposition of Co-Deposited Substrate and Plasma Polymer Coatings

### Materials and Methods

Films consisting of graded mixtures of stainless steel and plasma deposited carbon containing material were deposited by sputtering in argon gas containing a variable amount of acetylene gas. The conditions of deposition were as follows.

DC magnetron sputtering from a cylindrical stainless steel target (316 alloy) was used with a magnetic field applied from inside the cylinder by permanent magnets. The DC voltage was 650 V and the current was 3.5 A. The stainless steel target was 1.8 metres long and 80 mm diameter. The total pressure of argon and acetylene was maintained at approximately 1.0 Pa. The acetylene flow rate was varied throughout the deposition from zero to the maximum value desired. A PIII treated polyethylene surface used as a control.

The adhesion of the deposited surfaces was tested using tensile testing as described in Example 12.

The surfaces were incubated in HRP solution and the TMB assay was used to assess the activity of the surface attached enzyme as discussed in Example 12. The activity of the surfaces was measured as a function of time in buffer solution with periodic refreshing of the buffer.

The surfaces were incubated with tropoelastin solution and the presence of tropoelastin on the surfaces were analysed using an ELISA assay as described in Example 12. The tropoelastin treated surfaces were washed in SDS to assess the degree to which the attachment was covalent.

### Results and Discussion

The tensile adhesion strength of the plasma deposited graded layer was determined to be greater than 25 MPa. Failure occurred in the adhesive used to secure the samples onto the testing instrument rather than anywhere within the sample.

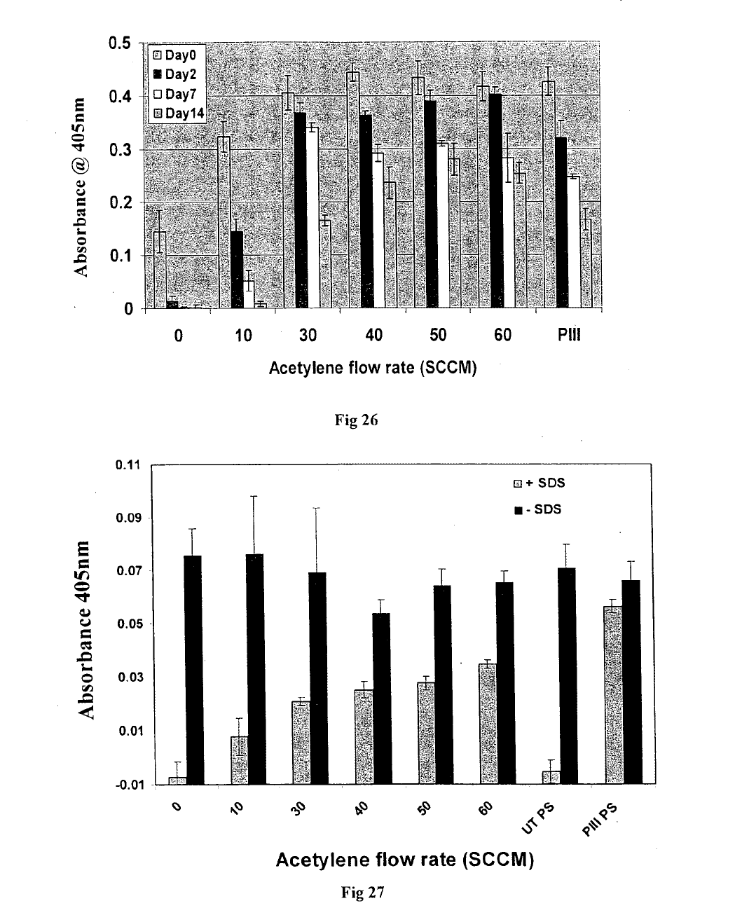

FIG. 26 shows the activity of HRP at days 2, 7 and 14 after incubation as a function of the final acetylene flow rate used during the deposition. The results show that the highest acetylene flow rates give the highest level of activity and activity retention. FIG. 27 shows the degree to which the binding of tropoelastin is SDS resistant. The degree of covalent binding can be seen to increase as the final acetylene flow rate used in the deposition increases.

### Conclusions

The use co-deposition to achieve a graded layer results in extremely strong adhesion of the deposited layer. Surfaces co-deposited from sputtered stainless steel and acetylene plasma show that there is a strong correlation between the flow rate of acetylene and the ability to attach protein covalently and to retain its biological function. The highest levels of both covalent attachment and retention of function were observed in the surfaces with the highest carbon content. We therefore infer that graded layers terminating in a high level of organic content and minimal level of stainless steel content provide the best platforms for protein attachment with adhesion sufficient for in vivo applications.

It is to be understood that the present invention has been described by way of example only and that modifications and/or alterations thereto, which would be apparent to a person skilled in the art based upon the disclosure herein, are also considered to fall within the scope and spirit of the invention, as defined in the appended claims.

### REFERENCES

1. S. Aibara, H. Yamashita, E. Mori, M. Kato, and Y. Morita, “Isolation and characterization of five neutral isoenzymes of horseradish peroxidase”, J Biochem (Tokyo), 1982 (92): 531-539.
2. G. Cohen, M. Kim and V. Oguwu, “A modified catalase assay suitable for a plate reader and for the analysis of brain cell cultures”, Journal of Neuroscience Methods, 1996, (67): 53-56.
3. Schena, M.; Shalon, D.; R. W. Davis; P. O. Brown Quantitative Monitoring of Gene Expression Patterns with a Complementary DNA Microarray Science 1995, 270, 467-470.
4. Kamal, J. K. A.; Behere, D. V. Thermal and conformational stability of seed coat soybean peroxidase Biochemistry 2002, 41, 9034-9042.
5. Wu W J, Vrhovski B, Weiss A S; J. Cell Biol. (1999) 274, 21719-21724
6. Stone P J, Morris S M, Griffin S, Mithieux S, Weiss A S; Am. J. Respir. Cell Mol. Biol. (2001) 22, 733-9.
7. B. K. Gan, A. Kondyurin, M. M. M. Bilek, Langmuir, 2007, 23, 2741.
8. N. J. Nosworthy, J. P. Y. Ho, A. Kondyurin, D. R. McKenzie, M. M. M. Buick, Acta Biomaterialia, 2007, 3, 695.
9. An Introduction to Tissue-Biomaterial Interactions, K. C. Dee, D. A, Puleo, Wiley, Rena Bizios, 2002.
10. Adam W. Clarke, Eva C. Arnspang, Suzanne M. Mithieux, Emine Korkmaz, Filip Braet, and Anthony S. Weiss, Biochemistry 2006, 45, 9989-9996.
11. Stephen L. Martin, Bernadette Vrhovski and Anthony S. Weiss, Gene, 154 (1995) 159-166.

1. An activated metallic, semiconductor, polymer, composite and/or ceramic substrate, the substrate being bound through a mixed or graded interface to a hydrophilic plasma polymer surface that is activated to enable direct covalent binding to a functional biological molecule, the plasma polymer surface comprising a sub-surface that includes a plurality of cross-linked regions that are located from about 0.3 nm to about 1000 nm beneath the plasma polymer surface.

2. (canceled)

3. A device comprising an activated metallic, semiconductor, polymer, composite and/or ceramic substrate, the substrate being bound through a mixed or graded interface to a hydrophilic plasma polymer surface that is activated to enable direct covalent binding to a functional biological molecule, the plasma polymer surface comprising a sub-surface that includes a plurality of cross-linked regions that are located from about 0.3 nm to about 1000 nm beneath the plasma polymer surface.

4. (canceled)

5. The activated substrate of claim 1, wherein the substrate comprises metal.

6. The activated substrate of claim 1, wherein the substrate comprises metal alloy.

7. The activated substrate of claim 5, wherein the metal comprises iron, copper, zinc, lead, aluminium, titanium, gold, platinum, silver, cobalt, chromium, vanadium, tantalum, nickel, magnesium, or manganese.

8. The activated substrate of claim 6, wherein the metal alloy is cobalt chrome, nickel titanium, titanium vanadium aluminium, or stainless steel.

9. The activated substrate of claim 1, wherein the substrate comprises a semiconductor.

10. The activated substrate of claim 9, wherein the semiconductor comprises silicon, germanium, gallium arsenide, indium antimonide, diamond, amorphous carbon, or amorphous silicon.

11. The activated substrate of claim 1, wherein the substrate comprises ceramic.

12. The activated substrate of claim 11, wherein the ceramic comprises an oxide, a silicate, a silicide, a nitride, a carbide or a phosphate.

13. The activated substrate of claim 11, wherein the ceramic comprises magnesium oxide, aluminium oxide, hydroxyapatite, titanium nitride, titanium carbide, aluminium nitride, silicon oxide, zinc oxide, or indium tin oxide.

14. The activated substrate of claim 1, wherein the substrate comprises a composite material.

15. The activated substrate of claim 14, wherein the composite material comprises a metal ceramic composite, an oxide composite or a ceramic polymer composite.

16. The activated substrate of claim 1, wherein the plasma polymer is generated from monomer units selected from n-hexane, allylamine, acetylene, ethylene, methane, and ethanol.

17. The activated substrate of claim 1, wherein the plasma polymer surface has a thickness of between about 0.3 nm to about 1000 nm.

18. (canceled)

19. The activated substrate of claim 1, wherein the mixed or graded interface is generated by varying composition of gases supplied to the process chamber so that deposited and/or implanted material changes progressively from more metallic to more polymeric.

20. The activated substrate of claim 1, wherein presence of the plurality of sub-surface cross-linked regions results in delay of plasma polymer surface hydrophobic recovery.

21. The activated substrate of claim 1, wherein the biological molecule comprises one or more amino acids, peptides, proteins, glycoproteins, lipoproteins, nucleotides, oligonucleotides, nucleic acids, lipids and/or carbohydrates.

22. The activated substrate of claim 1, wherein the biological molecule comprises a drug or drug target.

23. The activated substrate of claim 1, wherein the biological molecule comprises one or more of antibodies, immunoglobulins, receptors, enzymes, neurotransmitters, cytokines, hormones, complimentarity determining proteins, DNA, RNA and active fragments thereof.

24. The activated substrate of claim 1, wherein the biological molecule comprises one or more molecules that are integral to or attached to cells or cellular components.

25. The activated substrate of claim 1, wherein the substrate takes the form of a block, sheet, film, tube, strand, fibre, piece or particle, powder, shaped article, woven fabric or massed fibre pressed into a sheet.

26. The device of claim 3, which is, or is a component of, a diagnostic kit, a biosensor, a fuel cell or device for chemical processing, a cell or tissue culture scaffold, an analytical plate, an assay component or a medical device.

27. The device of claim 3, which is, or is a component of, a medical device.

28. The medical device of claim 27 selected from a contact lens, a stent, a pace maker, a hearing aid, a prosthesis, an artificial joint, a bone or tissue replacement material, an artificial organ, a heart valve, a replacement vessel, a suture, staple, nail, screw, bolt or other device for surgical use or other implantable device.

29. (canceled)

30. (canceled)

31. An activated metallic, semiconductor, polymer, composite and/or ceramic substrate, the substrate being bound through a mixed or graded interface to a hydrophilic plasma polymer surface that is activated to enable direct covalent binding to a functional biological molecule, the plasma polymer surface comprising a sub-surface that includes a plurality of cross-linked regions that are located from about 0.3 nm to about 1000 nm beneath the plasma polymer surface, the activated substrate produced according to a method comprising exposing a surface of the substrate to any or more of (i) to (iii):
(i) plasma ion implantation with carbon containing species;
(ii) co-deposition under conditions in which substrate material is deposited with carbon containing species while gradually reducing substrate material proportion and increasing carbon containing species proportion;
(iii) deposition of a plasma polymer surface layer with energetic ion bombardment.

32. (canceled)

33. (canceled)
